(19) United States
(12) Patent Application Publication
(10) Pub. No.: US 2026/0080854 A1
(43) Pub. Date: Mar. 19, 2026

(54) SYSTEMS AND METHODS FOR ATTENUATING ACOUSTIC WAVES

(71) Applicant: The General Hospital Corporation, Boston, MA (US)

(72) Inventors: Seok-Hyun Yun, Cambridge, MA (US); Soroush Shabahang, Cambridge, MA (US)

(21) Appl. No.: 19/109,980

(22) PCT Filed: Sep. 8, 2023
(86) PCT No.: PCT/US2023/073768

§ 371 Date: Mar. 7, 2025

Related U.S. Application Data
(60) Provisional application No. 63/374,924, filed on Sep. 8, 2022.

(51) Int. Cl. G10K 11/04; A61F 11/14; H04R 1/10

(52) U.S. Cl. CPC G10K 11/04; H04R 1/1083; A61F 11/145

(57) ABSTRACT

An acoustic filter can include a first substrate including a first plurality of holes directed, therethrough, a second substrate including a second plurality of holes directed therethrough, a chamber defined between the first substrate and the second substrate, and a membrane positioned within the chamber. The membrane can have a dimension other than the thickness of the membrane that can be less than a corresponding dimension of the chamber. The acoustic filter can be configured to attenuate a first acoustic wave that passes through the acoustic filter, the first acoustic wave can have a first amplitude above an amplitude threshold. The acoustic filter can be configured to passthrough a second acoustic wave without substantially attenuating the second acoustic wave, the second acoustic wave can have a second amplitude below’ the amplitude threshold.

### CROSS-REFERENCE TO RELATED APPLICATIONS

This application claims priority to U.S. Patent Application No. 63/374,924 filed Sep. 8, 2022, and entitled, “Devices for Controlling Acoustic Wave Transmission,” which is hereby incorporated by reference in its entirety.

### STATEMENT REGARDING FEDERALLY SPONSORED RESEARCH

This invention was made with government support under FA9550-17-1-0277 and FA9550-20-1-0063, awarded by the Air Force Office of Scientific Research (“AFOSR”). The government has certain rights in the invention.

### BACKGROUND

Noise-induced hearing loss is a widespread problem. According to Occupational Safety and Health Administration (“OSHA”) in U.S. Department of Labor, sounds above 85 dBA sound pressure level (“SPL”) can lead to gradual hearing loss if exposed for more than 8 hours a day. The allowed time of exposure for continuous noises is reduced by half for each 5 dB increase of SPL. Workers are not allowed to be exposed to any continuous noises above 115 dB without hearing protection. Exposure to impulsive or impact noise should not exceed 140 dB peak pressure, a typical pain threshold. A typical gunfire can impose 155-160 dB impulse noise to the shooter. Even a single blast, when transmitted to the inner ear, can cause instant and irreversible damage to hair cells. Warfighters and other supporting personnel (e.g., medics) are constantly exposed to the high risk of blast-induced hearing loss, making hearing loss one of the most prevalent service connected disabilities. As of 2020, more than 1.3 million veterans in the United States received disability compensation for hearing loss. Therefore, noise-induced hearing loss continues to be a problem for United States veterans, even aside from the great number of other individuals with jobs, activities, etc., which expose them to this type of hearing loss. Thus, it would be desirable to have improved systems and methods for attenuating acoustic waves.

### SUMMARY OF THE DISCLOSURE

Some non-limiting examples of the disclosure provide an acoustic filter. The acoustic filter can include a first substrate including a first plurality of holes directed therethrough, a second substrate including a second plurality of holes directed therethrough, a chamber defined between the first substrate and the second substrate, and a membrane positioned within the chamber. The membrane can have a dimension other than the thickness of the membrane that can be less than a corresponding dimension of the chamber. The acoustic filter can be configured to attenuate a first acoustic wave that passes through the acoustic filter, the first acoustic wave can have a first amplitude above an amplitude threshold. The acoustic filter can be configured to passthrough a second acoustic wave without substantially attenuating the second acoustic wave, the second acoustic wave can have a second amplitude below the amplitude threshold.

In some non-limiting examples, a gap is between a peripheral end of a membrane and a chamber.

In some non-limiting examples, a gap can be between a peripheral end of a membrane and a top of the chamber. In some non-limiting examples, a gap can be between the peripheral end of the membrane and a bottom of the chamber.

In some non-limiting examples, a dimension of a membrane other than a thickness of the membrane is a length, a width, or a diameter.

In some non-limiting examples, a membrane can have a length that can be less than a length of the chamber, such that a first longitudinal end of the membrane does not contact a first substrate or a second substrate.

In some non-limiting examples, a second longitudinal end of a membrane opposite a first longitudinal end of the membrane does not contact a first substrate or a second substrate.

In some non-limiting examples, a length of a membrane can be smaller than a length of a first substrate. The length of the membrane can be smaller than a length of a second substrate.

Some non-limiting examples of the disclosure provide an acoustic filter. The acoustic filter can include a first substrate including a first plurality of holes directed therethrough, a second substrate including a second plurality of holes directed therethrough, a chamber defined between the first substrate and the second substrate, and a membrane positioned within chamber. The membrane can be unconstrained or can be not taut between opposing ends of the membrane prior to an acoustic wave being applied to the acoustic filter. The acoustic filter can be configured to attenuate a first acoustic wave that passes through the acoustic filter, the first acoustic wave can have a first amplitude above an amplitude threshold. The acoustic filter can be configured to passthrough a second acoustic wave without substantially attenuating the second acoustic wave, the second acoustic wave can have a second amplitude below the amplitude threshold.

In some non-limiting examples, a gap can be between a top of a membrane and a top of a chamber. A gap can be between a bottom of the membrane and a bottom of the chamber.

In some non-limiting examples, a first end of the membrane can be not coupled to the first substrate or the second substrate, a second end of the membrane opposite the first end can be not coupled to the first substrate or the second substrate, and a center of the membrane can be between the first end and the second end can be not coupled to the first substrate or the second substrate.

In some non-limiting examples, a membrane can be free to translate along a longitudinal axis of the acoustic filter that is substantially parallel to a longitudinal dimension of the membrane.

In some non-limiting examples, a membrane can be unconstrainted or not taut can avoid substantially adding an acoustic impedance to an acoustic filter that results in acoustic losses for sound transmitted therethrough.

In some non-limiting examples, an acoustic filter can be configured to passively attenuate a first acoustic wave, such that no electrical power source is needed.

In some non-limiting examples, each hole of a first plurality of holes can be aligned with a respective hole of a second plurality of holes.

In some non-limiting examples, a chamber can include a first width at a first end of the chamber, a second width at a second end of the chamber, and a third width at a center of the chamber. The first width, the second width, and the third width can be substantially the same.

In some non-limiting examples, a cross-section of a chamber can be rectangular or square.

In some non-limiting examples, a width of the chamber can be substantially uniform along an entire length of the chamber.

In some non-limiting examples, a first amplitude of a first acoustic wave can be greater than or equal to 70 dB. A second amplitude of a second acoustic wave can be less than 70 dB.

In some non-limiting examples, a first amplitude of a first acoustic wave can be greater than or equal to 120 dB.

In some non-limiting examples, a first amplitude of a first acoustic wave can be greater than or equal to 150 dB. The first amplitude of the first acoustic wave can be greater than or equal to 170 dB.

In some non-limiting examples, a width of a chamber can be less than 3 millimeters, 2 millimeters, 1 millimeter, or 0.6 millimeters.

In some non-limiting examples, each hole of a first plurality of holes can have a size that can be less than or equal to 7.5 millimeters or 0.5 millimeters. Each hole of a second plurality of holes can have a size that can be less than or equal to 7.5 millimeters or 500 millimeters.

In some non-limiting examples, a first substrate can be rigid or semi-rigid. A second substrate can be rigid or semi-rigid.

In some non-limiting examples, a first amplitude of a first acoustic wave can be attenuated by at least 30 dB.

In some non-limiting examples, a first substrate can include a first recess fluidly coupled to a first plurality of holes. A second substrate can include a second recess fluidly coupled to a second plurality of holes. The first recess and the second recess can define the chamber.

In some non-limiting examples, a membrane does not include a hole directed therethrough. The membrane can have a planar surface, such that the membrane can be substantially flat. The membrane can have a surface that can be substantially smooth.

Some non-limiting examples of the disclosure provide an acoustic filter. The acoustic filter can include a first substrate including a first plurality of holes directed therethrough, a second substrate including a second plurality of holes directed therethrough, and a chamber defined between the first substrate and the second substrate. The chamber can have a first width at a top of the chamber and a second width at a center of the chamber. The first width and the second width can be substantially the same. The acoustic filter can include a membrane positioned within the chamber. The acoustic filter can be configured to attenuate a first acoustic wave that passes through the acoustic filter, the first acoustic wave can have a first amplitude above an amplitude threshold. The acoustic filter can be configured to passthrough a second acoustic wave without substantially attenuating the second acoustic wave, the second acoustic wave can have a second amplitude below the threshold.

In some non-limiting examples, each hole of a first plurality of holes and a second plurality of holes can have a size that is less than 0.5 millimeters.

In some non-limiting examples, a first width of a chamber can be less than or equal to 300 μm. The second width of the chamber can be less than or equal to 300 μm.

Some non-limiting examples of the disclosure provide a hearing protection device. The hearing protection device can include a body that can be configured to be placed over an ear or placed into an ear canal of the ear, and an acoustic filter coupled to or positioned within the body. The acoustic filter can be configured to be fluidly coupled to the canal of the ear. The acoustic filter can be configured according to any of the acoustic filters described herein.

Some non-limiting examples of the disclosure provide a sensor assembly. The sensor assembly can include an acoustic transducer that can be configured to convert acoustic waves into electrical signals, and an acoustic filter fluidly coupled to the acoustic transducer. The acoustic filter can be positioned in front of the acoustic transducer, such that an acoustic wave propagates first through the acoustic filter and then to the acoustic transducer. The acoustic filter can include a first substrate including a first plurality of holes directed therethrough, a second substrate including a second plurality of holes directed therethrough, a chamber defined between the first substrate and the second substrate, and a membrane positioned within chamber. The acoustic filter can be configured to attenuate a first acoustic wave that passes through the acoustic filter, the first acoustic wave can have a first amplitude above an amplitude threshold. The acoustic filter can be configured to passthrough a second acoustic wave without substantially attenuating the second acoustic wave, the second acoustic wave can have a second amplitude below the amplitude threshold.

In some non-limiting examples, an acoustic transducer can be a microphone sensor. The sensor assembly can be a microphone.

Some non-limiting examples of the disclosure provide a hearing aid. The hearing aid can include any sensor assembly described herein.

Some non-limiting examples of the disclosure provide a hearing device. The hearing device can include an acoustic transducer that can be configured to convert electrical signals into acoustic waves, and an acoustic filter configured to be fluidly coupled to an ear canal. The acoustic filter can include a first substrate including a first plurality of holes directed therethrough, a second substrate including a second plurality of holes directed therethrough, a chamber defined between the first substrate and the second substrate, and a membrane positioned within chamber. The acoustic filter can be configured to attenuate a first acoustic wave that passes through the acoustic filter, the first acoustic wave can have a first amplitude above an amplitude threshold. The acoustic filter can be configured to passthrough a second acoustic wave without substantially attenuating the second acoustic wave, the second acoustic wave can have a second amplitude below the threshold.

In some non-limiting examples, an acoustic filter can be positioned in front of an acoustic transducer, such that an acoustic wave generated from the acoustic transducer propagates through the acoustic filter and then to into the ear canal. In some configurations, the acoustic filter can be positioned behind the acoustic transducer, such that an acoustic wave generated from the acoustic transducer avoids passing through the acoustic filter before entering the ear canal.

In some non-limiting examples, a hearing device can be a hearing aid.

Some non-limiting examples of the disclosure provide an acoustic filter. The acoustic filter can include a first substrate including a first plurality of holes directed therethrough, a second substrate including a second plurality of holes directed therethrough, and a membrane positioned between the first substrate and the second substrate. The membrane can have a dimension other than the thickness of the membrane that can be less than a corresponding dimension of the first substrate or the second substrate. The acoustic filter can be configured to attenuate a first acoustic wave that passes through the acoustic filter, the first acoustic wave having a first amplitude above an amplitude threshold.

In some non-limiting examples, an acoustic filter can be configured to passthrough a second acoustic wave without substantially attenuating the second acoustic wave, the second acoustic wave can have a second amplitude below the amplitude threshold.

In some non-limiting examples, an amplitude threshold is less than or equal to 115 dB.

Some non-limiting examples of the disclosure provide an acoustic device in accordance with any non-limiting example disclosed herein, alone or in combination with any other device or system.

Some non-limiting examples of the disclosure provide a method of manufacturing any of the acoustic devices, acoustic filters, etc., described herein.

Some non-limiting examples of the disclosure provide a method of treating a patient using any of the acoustic devices, acoustic filters, etc., described herein, alone or in combination

with any other method.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The following drawings are provided to help illustrate various features of non-limiting examples of the disclosure, and are not intended to limit the scope of the disclosure or exclude alternative implementations.

FIG. 1A shows a schematic illustration of an isometric view of an acoustic filter.

FIG. 1B shows a top view of the acoustic filter of FIG. 1A.

FIG. 2A shows an exploded view of the acoustic filter of FIG. 1A.

FIG. 2B shows side views of the substrates and the membrane of the acoustic filter of FIG. 1A.

FIG. 2C shows a schematic illustration of a top view of another substrate.

FIG. 3 shows a cross-sectional view of the acoustic filter of FIG. 1A taken along lines 3-3 of FIG. 1B.

FIG. 4 shows the cross-sectional view of the acoustic filter of FIG. 3 in two different attenuating configurations.

FIG. 5 shows a schematic illustration of various different devices.

FIG. 6 shows a schematic illustration of the incorporation of multiple filters for devices.

FIG. 7 shows schematic illustrations of specific implementations of the devices shown in FIG. 6.

FIG. 8 shows an example illustration of earplugs that are enlarged relative to the individual to highlight features of the earplugs.

FIG. 9 shows a flowchart of a process for selectively attenuating acoustic waves.

FIG. 10 shows a flowchart of a process for manufacturing an acoustic filter and can include a method of manufacturing a hearing protection device or other devices described herein.

FIG. 11 shows a front view and a side view of a grid of the filter, along with a cross-section of an assembled acoustic filter as well as an exploded view of an assembled acoustic filter.

FIG. 12 shows schematic illustrations of the membrane of the acoustic filter in different positions and a graph illustrating the non-linear attenuation profile of the acoustic filter.

FIG. 13 shows a graph of the attenuation of different acoustic filters having different sized holes.

FIG. 14 shows a graph of the frequency response for grids of different thicknesses.

FIG. 15 shows a graph of the frequency response for grids of different thicknesses.

FIG. 16 shows a graph of the frequency response for grids having different filling factors.

FIG. 17 shows a schematic illustration of different shapes for the holes of the grids.

FIG. 18 shows a frequency response for membranes having different thicknesses and materials.

FIG. 19 shows a graph of the frequency response for membranes having different properties.

FIG. 20 shows a schematic illustration of a front view of a grid of an assembled acoustic filter, a side view of the assembled filter, and a cross-sectional view of the assembled filter.

FIG. 21 shows schematic illustration of various hearing protection devices that can include an acoustic filter.

FIG. 22 shows an illustration of an earmuff prototype with multiple acoustic filters distributed within an ear cover.

FIG. 23 shows a schematic illustration of a top view of an earmuff including multiple acoustic filters.

FIG. 24 shows a schematic illustration of a top view of another earmuff including multiple acoustic filters and a tilted or curved cap that can function as an acoustic reflector.

FIG. 25 shows a schematic illustration of a top view of another earmuff including multiple acoustic filters that are tilted relative to the user.

FIG. 26 shows an experimental set-up for testing the acoustic filter.

FIG. 27 shows an experimental setup for testing the acoustic filter for high-SPL input impulses.

FIG. 28 shows an experimental setup to test an earmuff prototype that includes an acoustic filter.

FIG. 29 shows a typical recordings of a filter with 10-μm-thick HDPE membrane and 30 μm gap size.

FIG. 30 shows the spectral transmission loss for the filter that was used for the data in FIG. 29.

FIG. 31 shows the attenuation of 10s of impulses with different SPL peak values after passing through a filter with similar parameters of the filter used in FIGS. 29 and 30.

FIG. 32 shows two possible forms of the grid pairs.

FIG. 33 shows an acoustic filter prior to coupling the substrates together.

FIG. 34A shows a schematic of the SHIELD filter.

FIG. 34B shows the membrane's free vibration for a low-SPL input.

FIG. 34C shows the membrane's obstructed vibration for a high-SPL impulse above the threshold.

FIG. 34D shows a schematic of the expected nonlinear transmission curve with a sharp transition at the threshold and increasing attenuation above the threshold.

FIG. 34E shows a graph of the vibration amplitude of the air by SPL for three different frequencies.

FIG. 34F shows a graph of the threshold SPL for monotonic waves of different frequencies for various gap sizes G−L₀.

FIG. 34G shows a graph of the restriction-induced loss as a function of peak SPL at 2 kHz for different gap sizes.

FIG. 34H shows a graph of the input-output curves from the nonlinear attenuation in FIG. 34G.

FIG. 35A shows four different design parameters.

FIG. 35B shows theoretical plots of transmission for different membrane thicknesses.

FIG. 35C shows theoretical plots of transmission for different hole filling ratios for a grid size of 50 μm.

FIG. 35D shows theoretical plots of transmission for different grid lengths for a hole filling ratio of 0.2.

FIG. 35E shows theoretical plots for different gap size for a grid size of 50 μm and filling ratio of 0.2.

FIG. 35F shows illustrations of an aluminum membrane, a grid, an assembled filter, and the transmission measurement setup for white noise at 90 dB SPL.

FIG. 35G shows the experimental results for different thicknesses of membrane placed between grids (Lₕ=1 mm, S=20%, G=50 μm, D₀=15 mm).

FIG. 35H shows the experimental results for different hole filling ratios.

FIG. 35I shows the experimental results for different grid lengths.

FIG. 35J shows the experimental results for different grid separations G.

FIGS. 36A-E show impulse responses measured experimentally from 5 samples. The light curves represent input blasts recorded by the external microphone, while the darker curves represent transmitted signals recorded by the internal microphone. The first and second graphs for each FIG. 36 (e.g., FIG. 36A) show responses to lower peak SPLs (below threshold) in linear and dB scales, respectively. The third and fourth graphs for each FIG. 36 (e.g., FIG. 36A) display data for 160 dB peak SPL. Nonlinear attenuation is absent in FIG. 36A, is modest in FIG. 36B, but becomes increasingly evident as the gap size is reduced.

FIG. 37A shows input-output curves of 8 different devices with different gap sizes.

FIG. 37B shows various graphs. The first graph of FIG. 37B shows a measured threshold SPL values for different gap sizes. The second and third graphs of FIG. 37B show attenuation measured as a function of input SPL for two representative devices in FIG. 37A.

FIG. 38A shows an illustration of a prototype earplug and the measurement setup using a model ear.

FIG. 38B shows a graph of a comparison of experimentally measured attenuation of the earplug (open circles) within the ear model and the performance of the bare filter itself (solid circles).

FIGS. 38C-E show time-domain recordings of the incoming and transmitted pressure levels for FIG. 38C high-, FIG. 38D moderate-, and FIG. 38E low-SPL blasts.

FIG. 39A shows an illustration of the prototype.

FIG. 39B shows transmission profiles of the 7 individual filters tested separately against 90 dB white noise (i.e., upper) and 160 dB blasts (i.e., lower). Thicker curves represent the mean values of the 7 filters.

FIG. 39C shows the power spectra of the internal microphone compared between open ear (i.e., baseline), the SHIELD earmuff, and an unmodified commercial earmuff against 90 dB white noise.

FIG. 39D shows the transmission characteristic of the prototype earmuff.

FIG. 40A shows microphone data for a 700 Hz monotone input at 100 dB SPL (i.e., input).

FIG. 40B shows wind-induced attenuations measured for various input SPL and frequencies for wind blowing at a normal angle to the device at 26 km/h.

FIG. 40C shows attenuation at different wind speeds measured using white noise with 100 dB SPL.

### DETAILED DESCRIPTION OF THE PRESENT DISCLOSURE

As described above, noise-induced hearing loss is a widespread problem for United States military members and other supporting members (e.g., medics, diplomats, etc.). For example, typical gunfire, and other blasts in which these members and personnel are routinely exposed to, can exceed 140 dB, which can cause instant and irreversible damage to the hair cells, rupture the eardrum, etc. Aside from combat related noise-induced hearing loss, other non-combat situations can drive hearing loss in a similar manner. For example, police officers and other individuals (e.g., during recreational hunting) that discharge guns and other weapons, or that are in the vicinity of discharged weapons can sustain noise-induced hearing losses from these discharges. As another example, construction workers that operate heavy machinery (e.g., loaders, excavators, other heavy construction equipment), or machinists that operate mills, grinders, lathes, etc., are routinely exposed to loud noise impulses, which can cause noise-induced hearing losses from these impulses, especially over prolonged periods of time (e.g., during the span of a career). As yet another example, dentists and other medical professionals that operate drills during surgery (e.g., orthopedic surgeons), are also routinely exposed to loud noise impulses, such as when the drill contacts a particular tissue, and these loud noise impulses can cause noise-induced hearing losses for the dentists and other medical professionals including their supporting staff that are in the general vicinity (e.g., dental assistants, medical assistants, nurses, etc.). As a further example, everyday workers, commuters, etc., are also exposed to loud noise impulses from public transportation, planes (e.g., the sound of jet engines, such as a baggage handler at an airport), which can cause noise-induced hearing losses for those in a relatively close proximity to these noises. In addition to these examples, all these individuals are required to converse with others (e.g., particularly those in which the noises occur during a workday), or otherwise hear noises in their surroundings to accurately and safely orient themselves.

Some conventional approaches have attempted to address these issues. For example, users can wear conventional hearing protection devices, which can effectively reduce the risk of noise-induced hearing loss. However, these hearing protection devices have issues. For example, passive earplugs, such as foam earplugs can help reduce hearing loss (e.g., by attenuating sounds), but these fixed attenuation earplugs necessarily undesirably decrease the wearer's ability to hear normal sounds, such as conversations with others, and also make it difficult to determine the direction of sounds, which is especially crucial for situational awareness during military operations. Similarly, “combat” earplugs with sound-blocking caps can also help to reduce hearing loss, but require that the manual caps be closed. However, with the caps closed, wearers are limited in their ability to hear normal sounds including conversations, which could be at a fairly low decibel level (e.g., whispering) during military operations. Conversely, with the caps closed, military personnel can adequately hear natural noises, but are exposed to ear damage from unpredictable blasts, gunfire, etc. And due to the unpredictable nature of the blasts, gunfire, etc., wearers are unlikely to be able to put the caps back on in time, even assuming the situation permitted them to focus solely on putting the caps back on (e.g., an unexpected blast forces military personnel to focus on more important tasks, such as seeking safety). These limitations often lead to military personnel having poor compliance with hearing protection devices, choosing to avoid using hearing protection devices altogether.

Others have attempted to solve these problems, but without success. For example, conventional orifice filters and sound-induced movable plugs have shown SPL-dependent transmission, but have limited dynamic range and relatively high background attenuation. In fact, orifice earplugs have been used by the U.S. military, but nearly 300,000 military veterans have filed lawsuits alleging that the dual-ended Combat Arms earplugs were defective and failed to protect them from hearing damage. Other approaches involve active electronic hearing protection devices, which provide electronics-controlled variable transmission. However, these devices require power sources, are more expensive, and do not reproduce all the qualities of natural sound waves. This is especially problematic in combat roles, in which a dead battery making the device inoperable is undesirable (e.g., service personnel do not have time to replace these batteries in the field), and the inability to hear natural sounds limits sound recognition, sound localization, and spatial awareness.

As another example, the device described in U.S. Pat. No. 8,249,285 (“Method and Apparatus for Producing Nonlinear Sound Attenuation”) describes using a diaphragm positioned between two plates, in which the diaphragm is connected and held at its edges between the two plates. This constraining of the diaphragm undesirably introduces acoustics losses (e.g., by increased acoustic impedance) and adds unwanted diaphragm resonances (e.g., which can distort the sound transmitted to the person). In addition, the holes in the plates are described as being 66 micrometers in diameter, which may be too small and adds transmission losses for low frequencies due to viscous damping. Still further, the diaphragm is described as expanding and curving when sound pressure is applied, both of which are undesirable due to increasing transmission losses. In addition a curved diaphragm in contact with a curved plate is not only more difficult to manufacture, but also the curved diaphragm may not make flush abutment over the holes in the plate due to different heights of the plate, which can undesirably allow unwanted high pressure sound waves therethrough. In this case, the larger the curve of the diaphragm the greater the issues. Additionally, the diaphragm is described as only being made of flexible materials (and not elastic materials), which may not respond adequately to lower blast pressures, which are more typical than higher blast pressures.

As yet another example, U.S. Patent Application Publication 2017/0200440 (“Acoustic Attenuation Device and Methods of Producing Thereof”) describes a movable diaphragm supported by springs that anchor to a substrate, and the moveable diaphragm with at least one hole in it. Similarly to the device in described in U.S. Pat. No. 8,249,285, this moveable diaphragm is held and constrained. In addition, this moveable diaphragm does not protect from both the positive-pressure parts and the negative-pressure parts of a sound impulse because it lacks a second silicon nitride or polysilicon film.

Some non-limiting examples of the disclosure provide advantages to these issues (and others) by providing improved systems and methods for attenuating acoustic waves. For example, some non-limiting examples of this disclosure provide an acoustic filter that can include a first substrate with a first plurality of holes, a second substrate with a second plurality of holes, and a membrane positioned between the first substrate and the second substrate and positioned within a chamber. The membrane can be unconstrained or not taught prior to an acoustic wave being applied to the acoustic filter. In other words, the membrane is not held, coupled, connected, glued, or otherwise constrained by the substrates. In this way, the membrane is not tensilely loaded, such that no tensile strain is applied to the membrane. This freely moving membrane within the chamber avoids transmission losses for low amplitude acoustic waves (e.g., low SPL acoustic waves) and avoids introducing undesirable acoustic resonance attributable to the membrane. In addition, the first plurality of holes and the second plurality of holes are sized, such that they are not too small (e.g., increasing transmission losses) and are not too big (e.g., decreasing the effectiveness of the sound blocking for high pressure acoustic waves). Also, the width of the chamber is sized so that low pressure sounds are transmitted through with no or minimal transmission losses (e.g., without substantially attenuating an acoustic wave), while large pressure sounds are attenuated.

Non-limiting examples of the disclosure described herein can improve devices with microphones (e.g., hearing aids), speakers, ear protection devices or hearing protection devices (e.g., ear plugs, earmuffs, etc.), helmets, electronic devices (e.g., including those with fragile sensors), etc., by providing a non-linear acoustic filter that allows low SPL sound therethrough with minimal to no attenuation (e.g., sounds at conversational volumes), while attenuating sounds at high SPL levels (e.g., from blasts, gunfire, heavy machinery, machining tools, power tools, construction tools, etc.). In this way, an individual does not have to remove their ear protection device, or take the caps out, when conversing and hearing individuals, while at the same time protecting against unexpected loud noises (e.g., blasts, gunfire, etc.), in passive manner and nearly simultaneously and instantaneous. The present application has incorporated herein by reference in its entirety PCT application number PCT/US2017/040203 “Systems and Methods for Ear Protection.”

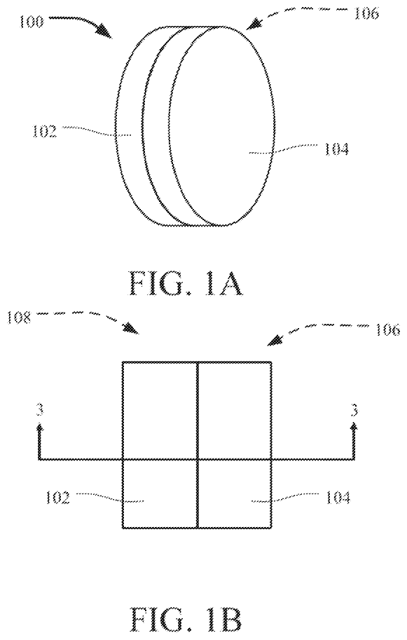

FIG. 1A shows a schematic illustration of an isometric view of an acoustic filter 100, while FIG. 1B shows a top view of the acoustic filter 100. The acoustic filter 100 can include substrates 102, 104, and a membrane 106 positioned between the substrates 102, 104. Although the substrates 102, 104 and the membrane 106 are illustrated as having the same shape (e.g., a peripheral shape), which in this case is a circle (e.g., or cylinder), the substrates 102, 104, and the membrane 106 can have other shapes such as a square, a rectangle, a triangle, prisms of these shapes, etc. The substrates 102, 104 can have the same outer width or diameter, such as shown in this case. In addition, the substrates 102, 104 can be substantially the same, or can be different. For example, the substrates 102, 104 can be identical, but mirror images of each other, or the substrate 102 can have an extension that engages with a relatively flat substrate 104 (or vice versa). The substrates 102, 104 can be rigid, or semi-rigid, which can prevent undesirable transmission losses at low SPL (e.g., acoustic waves with small amplitudes). For example, each substrate 102, 104 can have a modulus of rigidity that is within a range of about 1 MPa to about 100 MPa, a range of about 100 kPa to 10 GPa, etc. Thus, in some cases, each substrate 102, 104 can have a modulus of rigidity that is greater than about 100 kPa, less than about 10 GPa, etc.

The substrates 102, 104 can be formed out of different materials, which can be different from each other. For example, the substrates 102, 104 can be formed out of a metal, a polymer, a resin, etc. The substrate being formed out of a metal can be advantageous in that metals can be more structurally sound than other materials, metals have little to no acoustic absorption (e.g., low acoustic transmission losses), etc. In some cases, the substrates 102, 104 can be formed out of a plurality of tubes (e.g., metal tubes), which are coupled together. In this way, with the tubes, manufacturing of the substrate can be easier and more consistent (e.g., creating holes in a substrate may be difficult especially due to the small thickness of the substrate), the spatial layout of the holes can be uniform, and the filling factor (described in more detail below) can be advantageously minimized. In some cases, each substrate 102, 104 can have a thickness (e.g., between opposing lateral ends of a substrate at the recess of the substrate, or at the chamber defined at least partially by the substrate, or other portions of the substrate) that is less than or equal to about 8 millimeters, 7.5 millimeters, 6 millimeters, 5 millimeters, 2 millimeters, 1.2 millimeters, 1 millimeter, etc. In this way, with thinner substrates, higher frequency acoustic components are attenuated less than thicker substrates. In some cases, however, thicker substrates are desirable due to the frequency dependent attenuation especially at higher frequencies. In this case, each substrate 102, 104 can have a thickness that is greater than or equal to about 8 millimeters, 7.5 millimeters, 6 millimeters, 5 millimeters, 2 millimeters, 1.2 millimeters, 1 millimeter, etc. In this way, thicker substrates can be more robust at blocking the membrane and thus the acoustics waves transmitted therethrough, while at the same time attenuating higher frequency components that are largely absent from human speech (e.g., frequency components above 3 kHz).

The membrane 106 can be relatively thin so as to transmit acoustic waves through the acoustic filter 100 (e.g., thinner membranes have less mass and thus have less inertia thereby responding more quickly and stronger to the incoming acoustic wave with less distortion). Thus, the membrane 106 can have a thickness that is less than or equal to about 1 mm, 18 μm, 12.5 μm, 4 μm, 2.4 μm, 1.2 μm, etc. However, thinner membranes, especially those formed out of particular materials, may not be strong enough to survive a large amplitude acoustic wave (e.g., a high pressure acoustic wave). Thus, the membrane 106 can have a thickness that is greater than or equal to about 1 mm, 18 μm, 13 μm, 12.5 μm, 5 μm, 4.9 μm, 4 μm, 2.4 μm, 1.2 μm, etc. Similarly to the substrates 102, 104, the membrane 106 can be formed out of different materials. For example, the membrane 106 can be formed out of a polymer (e.g., high density polyethylene), a metal (e.g., copper, aluminum, etc.), etc. In some cases, the membrane 106 being formed out a of a metal can be advantageous in that metals have low acoustic absorption (e.g., low acoustic transmission losses), metals avoid being deformed inside holes when the membrane is forced against a substrate when there is a large acoustic pressure wave, and metals are generally stronger than other materials. In some specific cases, the membrane 106 can be formed out of copper, aluminum, titanium, iron, chromium, etc., which are generally lighter metals and are thus more responsive, while being largely inert (e.g., unlike alkaline metals). Although obscured from the view in FIG. 1A, but shown in other figures, the membrane 106 can be free of any holes directed therethrough. In this way, acoustic waves that are above a particular pressure threshold do not undesirably leak through and travel to the person's ear (or other instrument, detector, sensor, electronic component, etc., such as a microphone). In some non-limiting examples, although the membrane 106 is described as being a membrane, the membrane 106 can be a diaphragm, substrate, etc., that can transmit acoustic waves by vibrating (e.g., within the chamber of the acoustic filter) and can move to obstruct the transmission of acoustic waves that exceed an amplitude threshold, such as pressure (e.g., a positive threshold pressure or a negative threshold pressure).

Although not shown in FIG. 1A, but shown in other figures, the membrane 106 can be substantially flat and substantial smooth. For example, the membrane 106 can have a substantially (i.e., deviating from 30 percent from a) planar surface on a first lateral side of the membrane 106 and can have a substantially planar surface on a second lateral side of the membrane 106 opposite the first lateral sides, in which the first lateral side and the second lateral side of the membrane 106 define a thickness of the membrane 106. In some cases, this substantially planar surface can extend along the entire side of the membrane 106. In addition, these substantially planar surfaces can be substantially smooth. For example, the amplitude of the surface (e.g., in which the amplitudes define the vertical deviations of the roughness profile from the mean line), which can be the maximum amplitude of the surface (e.g., the maximum vertical deviation from the mean) can be less than or equal to about 1 μm, 100 nm, etc. In some cases, characteristics of the membrane 106 (e.g., the membrane not being curved or being planar) can occur before an acoustic wave is applied to the membrane 106, or after an acoustic wave is applied to the membrane 106. In the latter case, the membrane 106 not being curved while having acoustic waves applied thereto is better able to cover the holes of the substrate and attenuate the incoming acoustic wave. Similarly to the membrane 106, each substrate 102, 104 can have a substantially flat or planar surface, which can come into contact with the membrane 106 (or the membrane 106 covering the substantially flat surface). For example, the inner surface of each substrate 102, 104 at the respective recess can have the substantially flat or planar surface. This can ensure flush abutment between the membrane 106 and a substrate.

As shown in FIG. 1B, the substrates 102, 104 can flushly abut against each other, and can contain the membrane 106. In particular, the substrates 102, 104 can define a chamber 108 and the membrane 106 can be positioned within the chamber 108, which can include the membrane 106 being between the substrates 102, 104. In this way, the membrane 106 can be sealed within the chamber 108, such that dust, debris, etc., does not enter the chamber 108 over time, which may impede functionality of the membrane 106. Accordingly, the membrane 106 can be hermetically sealed within the chamber 108.

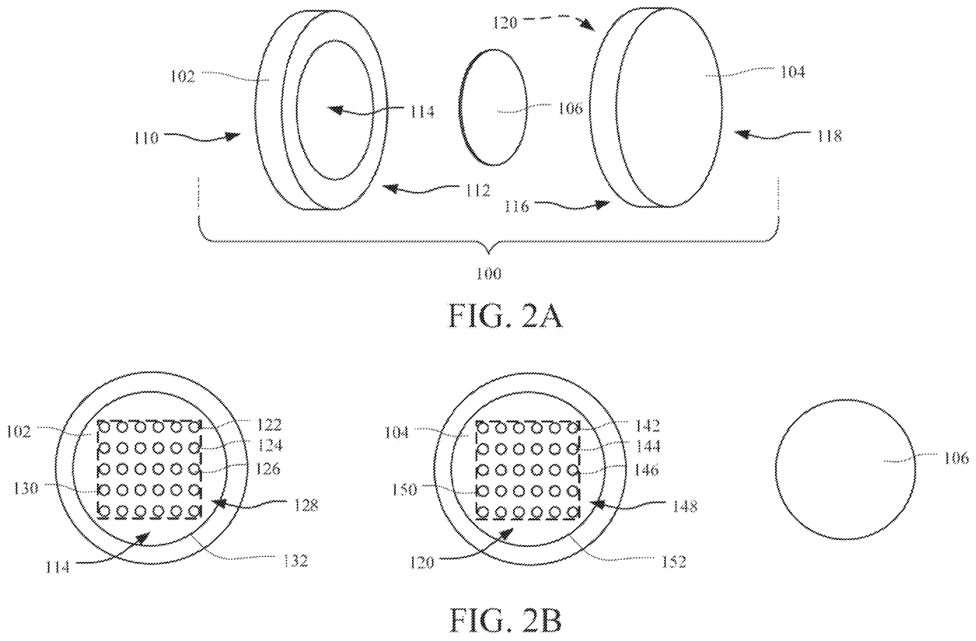

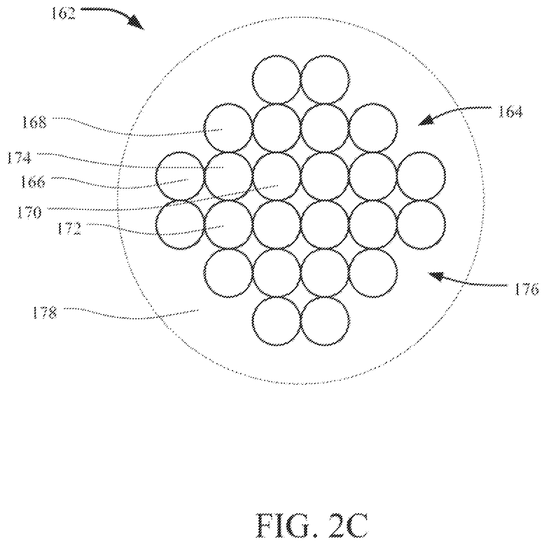

FIG. 2A shows an exploded view of the acoustic filter 100, while FIG. 2B shows side views of the substrates 102, 104, and the membrane 106. As shown in FIG. 2A, each substrate 102, 104 can have a recess, which when assembled together, can define the chamber 108. For example, the substrate 102 can have lateral sides 110, 112, in which the lateral side 110 is opposite the lateral side 112. The substrate 102 can include a recess 114 that is directed into the substrate 102 in a direction from the lateral side 112 towards the lateral side 110. Similarly, the substrate 104 can have lateral sides 116, 118, in which the lateral side 116 is opposite the lateral side 118. The substrate 104 can include a recess 120 that is directed into the substrate 102 in a direction from the lateral side 116 towards the lateral side 118. As described in more detail below, when the substrates 102, 104 are coupled together (e.g., with welding, metallic welding, ultrasonic welding, an adhesive such as a superglue, etc.), the membrane 106 can be positioned within the chamber 108 and sealed therein (e.g., in which the recesses 114, 120 can define the chamber 108). In particular, the membrane 106 can be positioned within either or both of the recesses 114, 120 of the substrates 102, 104 when the substrate 102, 104 are coupled together.

In some non-limiting examples, the membrane 106 can have the same shape (e.g., peripheral shape) as the chamber 108, and the recesses 114, 120. For example, the membrane 106 has a circular shape, and thus the chamber 108 and the recesses 114, 120 can also have a circular shape (e.g., a peripheral shape that is circular). In other cases, the membrane 106, the chamber 108, and the recess 114, 120 can have other shapes (e.g., a triangular shape, a square shape, etc.). In some cases, it is appreciated that although the shapes have been described with respect to the peripheral shape, the substrates 102, 104, the membrane 106, the chamber 108, and the recesses 114, 120 can have the same three-dimensional shape. As shown in FIG. 2A, the substrates 102, 104, the membrane 106, the chamber 108, and the recesses 114, 120 all have a cylindrical shape, however, each of these can have other three-dimensional shapes such as being a rectangular prism, a square prism, a triangular prism, etc. In some configurations, the membrane 106 can be thinner than each substrate 102, 104 (e.g., at the recess of a substrate).

As shown in FIG. 2A, the membrane 106 can have a dimension (other than its thickness) that is smaller than a corresponding dimension of each of the substrates 102, 104 and the chamber 108. For example, the membrane 106 can have a width (e.g., a diameter in the case of a circular shape) that is smaller than the width of each of the substrates 102, 104, and smaller than a width of the chamber 108, and thus smaller than the width of the recesses 114, 120. In this way, advantageously, the membrane 106 can be untethered, untaut (e.g., not tensilely loaded between the substrates 102, 104), unconstrained by the substrates 102, 104 (e.g., when acoustic pressure is applied to the membrane 106), does not expand (e.g., when an acoustic wave is applied thereto), etc., such that the membrane 106 is free to move within the chamber 108 thereby avoiding introducing unwanted resonant frequencies attributable to the membrane being tensilely loaded and attenuation losses at low acoustic pressures, which is described in more detail below.

As shown in FIG. 2B, the substrate 102 can include one or more holes directed therethrough. For example the substrate 102 can include holes 122, 124, 126 directed through the substrate 102. In some cases, the substrate 102 can include a plurality of holes 128 directed through the substrate 102, which can include two or more of the holes 122, 124, 126. As shown in FIG. 2B, the plurality of holes 128 can be arranged in an array on the substrate 102, in which the array can include a plurality of rows of holes and a plurality of columns of holes. In some cases, the plurality of holes 128 can define a perimeter 130, or in other words, an outer periphery of holes. The perimeter 130 or outer periphery of the plurality of holes 128 can have various shapes (e.g., a rectangle, a square, a triangle), and can have the same shape as the shape of the chamber 108 (and the recesses 114, 120). In this case, although the perimeter 130 is illustrated as being a square, the perimeter 130 can be a circle, so as to match with the circular peripheral shape of the chamber 108. Regardless of the configuration, the perimeter 130 of the plurality of holes 128 can be positioned within the recess 114, can overlap with a peripheral edge of the recess 114 or chamber 108, etc. In this way, all of the plurality of holes 128 can align with the chamber 108 and the recess 114.

As shown in FIG. 2B, each hole of the plurality of holes 128 can have the same width (e.g., at the center of the hole), the same diameter (e.g., when the hole is circular), the same dimension or size that is not the depth. In other cases, the plurality of holes 128 can have different widths, different diameters, etc. Similarly, FIG. 2B shows that the distance between adjacent holes of the plurality of holes 128 can be substantially the same (e.g., between the centers of the holes) for each pair of holes of the plurality of holes 128. In other configurations, adjacent holes of the plurality of holes 128 can have different spacings. Although FIG. 2B shows the plurality of holes 128 as being 30 holes, the plurality of holes 128 can have other numbers of holes, such as, greater than or equal to two, four, six, ten, 20, 30, 100, 200, etc. In some cases, each hole of the plurality of holes 128 can have a size (e.g., a width, such as a maximum width of the hole, a width at its center, diameter, etc.) that is less than or equal to about 10 millimeters, 5.8 millimeters, 5 millimeters, 2.5 millimeters, 0.6 millimeters, 0.3 millimeters, 0.2 millimeters, 0.17 millimeters, etc. In this way, with smaller holes, the membrane 106 is better able to block the holes and thus better attenuate the incoming acoustic wave (e.g., the membrane 106 is better able to cover smaller holes without being forced into the holes and vibrate therein). However, as the holes decrease in size, viscous damping increases, which undesirably introduces acoustic losses at low acoustic pressures, and also increases the likelihood of introducing high frequency acoustic resonances. Thus, in some cases, each hole of the plurality of holes 128 can have a size that is greater than or equal to about 10 millimeters, 5.8 millimeters, 5 millimeters, 2.5 millimeters, 0.6 millimeters, 0.3 millimeters, 0.2 millimeters, 0.17 millimeters, etc. In some specific cases, each hole of the plurality of holes 128 can have a size that is greater than about 0.170 millimeters, greater than about 0.270 millimeters, etc., which can avoid acoustic losses at low acoustic sound pressures.

In some cases, the substrate 102 can have a filling ratio, or in other words a hole filling ratio. The filling ratio can be defined as 1−area of the holes/total area, where the area of the holes is the collective area of the plurality of holes 128 within the perimeter 130, and were the total area is the total area of the inner surface 132 of the substrate 102 within the perimeter 130 including surfaces that have holes and that are free of holes (e.g., the area within the perimeter 130). A filling ratio of 1 means that there are no holes within the perimeter 130, while a filling ratio of 0 means that the substrate 102 has one hole that is the size of the perimeter 130. In some cases, the substrate 102 within the perimeter 130 can have a filing ratio that is greater than 0 and less than or equal to about 1, 0.95, 0.89, 0.74, 0.71, 0.69, 0.56, 0.5, 0.31, 0.25, 0.2, 0.14, 0.1, 0.02, etc. In some cases, it is desirable to have the filling ratio be as small as possible, within reason (e.g., without allowing the membrane to substantially deflect inside the holes), to avoid acoustic losses from low acoustic pressure waves. In some cases, including for smaller sized holes, decreasing filling factor below 0.1 may introduce acoustic attenuation losses at low acoustics pressures. Thus, the substrate 102 within the perimeter 130 can have a filing ratio that is greater than or equal to 0.02, 0.1, 0.25, etc. In some cases, the substrate 102 within the perimeter 130 can have a filing ratio that is about 0.5.

The substrate 104 can be implemented in a similar manner as the substrate 102. For example, the substrate 104 can include one or more holes directed therethrough. For example the substrate 104 can include holes 142, 144, 146 directed through the substrate 104. In some cases, the substrate 104 can include a plurality of holes 148 directed through the substrate 104, which can include two or more of the holes 142, 144, 146. As shown in FIG. 2B, the plurality of holes 148 can be arranged in an array on the substrate 104, in which the array can include a plurality of rows of holes and a plurality of columns of holes. In some cases, the plurality of holes 148 can define a perimeter 150, or in other words, an outer periphery of holes. The perimeter 150 or outer periphery of the plurality of holes 148 can have various shapes (e.g., a rectangle, a square, a triangle), and can have the same shape as the shape of the chamber 108 (and the recesses 114, 120). In this case, although the perimeter 150 is illustrated as being a square, the perimeter 150 can be a circle, so as to match with the circular peripheral shape of the chamber 108. Regardless of the configuration, the perimeter 150 of the plurality of holes 128 can be positioned within the recess 120, can overlap with a peripheral edge of the recess 120 or chamber 108, etc. In this way, all of the plurality of holes 148 can align with the chamber 108 and the recess 120.

As shown in FIG. 2B, each hole of the plurality of holes 148 can have the same width (e.g., at the center of the hole, at a maximum width of the hole, etc.), the same diameter (e.g., when the hole is circular), the same dimension or size that is not the depth. In other cases, the plurality of holes 148 can have different widths, different diameters, etc. Similarly, FIG. 2B shows that the distance between adjacent holes of the plurality of holes 148 can be substantially the same (e.g., between the centers of the holes) for each pair of holes of the plurality of holes 148. In other configurations, adjacent holes of the plurality of holes 148 can have different spacings. Although FIG. 2B shows the plurality of holes 148 as being 30 holes, the plurality of holes 148 can have other numbers of holes, such as, greater than or equal to two, four, six, ten, 20, 30, 100, 200, etc. In some cases, each hole of the plurality of holes 148 can have a size (e.g., a width, such as a maximum width of the hole, a width at its center, diameter, etc.) that is less than or equal to about 10 millimeters, 5.8 millimeters, 5 millimeters, 2.5 millimeters, 0.3 millimeters, 0.2 millimeters, 0.17 millimeters, etc. In this way, with smaller holes, the membrane 106 is better able to block the holes. However, as the holes decrease in size, viscous damping increases, which undesirably introduces acoustic losses at low acoustic pressures, and also increases the likelihood of introducing high frequency acoustic resonances. Thus, in some cases, each hole of the plurality of holes 148 can have a size that is greater than or equal to about 10 millimeters, 5.8 millimeters, 5 millimeters, 2.5 millimeters, 0.3 millimeters, 0.2 millimeters, 0.17 millimeters, etc. In some specific cases, each hole of the plurality of holes 148 can have a size that is greater than about 0.170 millimeters, greater than about 0.270 millimeters, etc., which can avoid acoustic losses at low acoustic sound pressures.

In some cases, the substrate 104 can have a filling ratio, or in other words a hole filling ratio. The filling ratio can be defined as 1−area of the holes/total area, where the area of the holes is the collective area of the plurality of holes 148 within the perimeter 150, and were the total area is the total area of the inner surface 152 of the substrate 104 within the perimeter 150 including surfaces that have holes and that are free of holes (e.g., the area within the perimeter 150). A filling ratio of 1 means that there are no holes within the perimeter 150, while a filling ratio of 0 means that the substrate 104 has one hole that is the size of the perimeter 150. In some cases, the substrate 104 within the perimeter 150 can have a filing ratio that is greater than 0 and less than or equal to about 1, 0.95, 0.89, 0.74, 0.71, 0.69, 0.56, 0.5, 0.31, 0.25, 0.2, 0.14, 0.1, 0.02, etc. In some cases, it is desirable to have the filling ratio be as small as possible, within reason (e.g., without allowing the membrane to substantially deflect inside the holes), to avoid acoustic losses from low acoustic pressure waves. In some cases, including for smaller sized holes, a decreasing filling factor below 0.1 may introduce acoustic attenuation losses at low acoustics pressures. Thus, the substrate 104 within the perimeter 150 can have a filing ratio that is greater than or equal to 0.02, 0.1, 0.25, etc. In some cases, the substrate 104 within the perimeter 150 can have a filing ratio that is about 0.5.

As shown in FIG. 2B, the membrane 106 can be free of any holes directed therethrough (e.g., the membrane 106 does not include a hole directed therethrough). In this way, the membrane 106 does not undesirably transmit high pressure acoustic waves through the hole in the membrane 106. In some non-limiting examples, the membrane 106 can have a substantially uniform thickness (e.g., the thickness does not substantially change along a dimension, such as the length or diameter of the membrane 106). Although not shown in FIG. 2B, when the acoustic filter 100 is assembled, the membrane 106 can extend across all the plurality of holes 128 of the substrate 102 and can extend across all the plurality of holes 148 of the substrate 104. In other words, the membrane 106 can extend beyond the perimeters 130, 150, or a peripheral end of the membrane 106 can overlap with each of the perimeters 130, 150.

FIG. 2C shows a schematic illustration of a top view of a substrate 162, which can be implemented in a similar manner as the substrates 102, 104. Thus, the description of the substrate 162 pertains to the substrates 102, 104 (and vice versa). Accordingly, either or both of the substrates 102, 104 can be implemented as described with respect to the substrate 162. The substrate 162 can include a plurality of tubes 164 that are coupled together to form an array of tubes. For example, each lateral side of each tube of the plurality of tubes 164 can be coupled to a respective lateral side of a respective tube of the plurality of tubes 164 adjacent the tube. More specifically, the plurality of tubes 164 can include tubes 166, 168, 170, 172, 174, in which the tubes 166, 168, 170, 172 can surround and can be coupled to the tube 174. The tube 174 can include four lateral sides and each of the four lateral sides of the tube 174 can be coupled to an adjacent lateral side of the tubes 166, 168, 170, 172. This pattern can propagate throughout all the plurality of tubes 164, with the understanding that not every lateral side of a tube may be coupled to a respective tube—especially at the outer periphery of the plurality of tubes 164 (e.g., the tubes that define the outer periphery of the array). Each tube of the plurality of tubes 164 can have a hole, such that the plurality of tubes 164 define a plurality of holes 176.

Although each tube of the plurality of tubes 164 is shown as being cylindrical and each hole of the plurality of holes 176 are shown as being circular in cross section (e.g., an axial cross section), each tube and each hole can have different shapes. For example, each tube of the plurality of tubes 164 can be a prism (e.g., a triangular prism, a square prism, a rectangular prism, an octagonal prism, etc.) or other three-dimensional shapes, while each hole of the plurality of holes 176 can have different cross sectional shapes other than circular, such as, for example, square, rectangular, triangular, octagonal, etc. Accordingly, each tube of the plurality of tubes 164 can have the same three-dimensional shape, and each hole of the plurality of holes 176 can have the same cross-sectional shape (e.g., an axial cross section). Similarly to the perimeter 130 of the substrate 102, the perimeter defined by the outer peripheral tubes of the plurality of tubes 164 can have different shapes (e.g., peripheral shapes), such as, for example, a square, a rectangle, a triangle, etc.

In some non-limiting examples, the substrate 162 can include a base 178 that can surround and can be coupled to the plurality of tubes 164, in a similar manner as the substrates 102, 104. In this way, the plurality of tubes 164 can define an array of holes, while the base 178 can support the plurality of tubes 164. In some cases, the base 178 can have a hole directed therethrough that can receive the plurality of tubes 164 that are coupled together, to assemble the substrate 162. In some cases, having the plurality of tubes 164 define the plurality of holes 176 (e.g., in an array) can be advantageous in that the assembly can be easier rather than drilling holes into thin plates, and the filling ratio can be desirably decreased without risk of destroying the thin substrate. For example, manufacturing holes into thin substrates can be difficult especially when they are close together (e.g., the regions between them may break). Alternatively, with the plurality of tubes, the thickness of each tube contributes little to the space without a hole, and the tubes can be more structurally sound. Thus, the plurality of tubes can advantageously minimize the filling ratio, while at the same time being more structurally sound. Although the base 178 is illustrated as having a shape (e.g., a peripheral shape) that is a circle, the base 178 can have other shapes (e.g., a circle, a square, a rectangle, etc.).

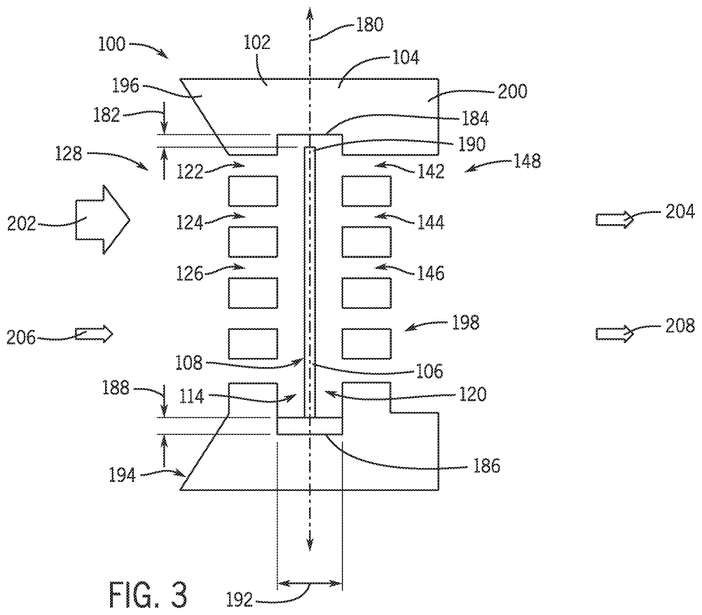

FIG. 3 shows a cross-sectional view of the acoustic filter 100 taken along lines 3-3 of FIG. 1B. This cross-sectional view is a frontal plane of the acoustic filter 100, with the top view of FIG. 1B being parallel to an axial plane of the acoustic filter 100. As shown in FIG. 3, the chamber 108 is positioned between the substrates 102, 104, and with the chamber 108 being positioned between the plurality of holes 128, 148. FIG. 3 also shows the membrane 106 being positioned entirely within the chamber 108 (e.g., the entire membrane 106 is positioned within the chamber 108). As described below, the membrane 106 being freely movable within the chamber 106 has advantages. In some cases, the recesses 114, 120 can define the chamber 108. For example, in this case, the recess 114 and the recess 120 can define equal portions of the chamber 108. However, in other configurations, the recess 114 can define more of the chamber 108 than the recess 114. In some cases, the recess 114 of the substrate 102 can define the entire width of the chamber 108, with the substrate 104 providing a wall that at least partially defines the chamber 108 (e.g., a wall of the chamber). In this case, the substrate 104 lacks the recess 120. Similarly, the recess 120 of the substrate 104 can define the entire width of the chamber 108, with the substrate 102 providing a wall that at least partially defines the chamber 108 (e.g., a wall of the chamber). In this case, the substrate 102 lacks the recess 114.

As shown in FIG. 3, each hole of the plurality of holes 128 can be aligned with a respective hole of the plurality of holes 148. For example, the holes 122, 142 can be aligned, the holes 124, 144 can be aligned, the holes 126, 146 can be aligned, etc. In some cases, and as shown in FIG. 3, the chamber 108 can extend past the holes that define the outer periphery of the plurality of holes 128 (e.g., the holes that exist on the periphery of the array) and can extend past the holes that define the outer periphery of the plurality of holes 148 (e.g., the holes that exist on the periphery of the array). For example, the chamber 108 can extend past (e.g., above) the holes 122, 142 positioned at an upper end of the plurality of holes 122 and the plurality of holes 142, in which the holes 122, 142 can be at least some of the highest holes of the plurality of holes 128 and the plurality of holes 148. As another example, the chamber 108 can extend past (e.g., below) holes of the substrates 102, 104 that can be at least some of the lowest holes of the plurality of holes 128 and the plurality of holes 148. In other words, a first portion of the chamber 108 can extend above the plurality of holes 128, 148, a second portion of the chamber 108 can extend below the plurality of holes 128, 148, a third portion of the chamber 108 can extend past (e.g., to a side) the plurality of holes 128, 148, and so on. Thus, the chamber 108 can have dimension (e.g., a length, a width, a diagonal, a diameter, etc.) that is larger than a corresponding dimension of the membrane 100, the plurality of holes 128, the perimeter 130, the plurality of holes 148, the perimeter 150, etc. For example, the chamber 108 can have a length (e.g., parallel to an axial axis 180 of the acoustic filter 100) that is longer than the length of the perimeter 130 of the plurality of holes 128, longer than the length of the perimeter 150 of the plurality of holes 148, etc. An example of this representation is shown in FIG. 2B, in which the recess 114 surrounds the plurality of holes 128, and in which the recess 120 surrounds the plurality of holes 148.

Similarly to the chamber 108 in which the membrane 106 is positioned, the membrane 100, such as a peripheral end of the membrane 100, can extend past the plurality of holes 128, 148 in multiple different directions (e.g., up, down, side, side, etc.). In some cases, this peripheral end 190 of the membrane 106 can surround the plurality of holes 128, 148 for a portion of the membrane 106 (e.g., a circumferential extent of the membrane 106) or for the entire membrane 106 (e.g., the circumferentially around the entire membrane 106). As described previously, the membrane 106 can have a dimension (e.g., length, width, diameter, diagonal) that is smaller than a corresponding dimension of each of the substrates 102, 104, and that is smaller than a corresponding dimension of the chamber 108 (including the recesses 114, 120. For example, a peripheral end 190 of the membrane 106 can be separated from the substrate 102 and the substrate 104. Accordingly, a gap 182 can be positioned between a peripheral end 190 of the membrane 106 and an end 184 (e.g., an upper end) of the chamber 108, which is opposite an end 186 of the chamber 108. In other words, the gap 182 can be positioned between the peripheral end 190 of the membrane 106 and the substrates 102, 104. Similarly, a gap 188 can be positioned between the peripheral end 190 of the membrane 106 and an end 186 (e.g., a lower end) of the chamber 108. In other words, the gap 186 can be positioned between the peripheral end 190 of the membrane 106 and the substrates 102, 104. In some cases, the gap 188 can extend partially or entirely around the membrane 106, chamber 108, the substrates 102, 104. In the case in which the gap 186 extends entirely around the membrane 106, the gaps 182, 186 can be the same size (e.g., the gaps 182, 186 are effectively the same). In other cases, the gaps 182, 188 can be different sizes. In some non-limiting examples, the peripheral end 190 of the membrane 106 can be positioned between the plurality of holes 128 (and the plurality of holes 148) and an end of the chamber 108, or stated another way, the peripheral end 190 of the membrane 106 can surround the plurality of holes 128 (and the plurality of holes 148) of the substrates 102, 104, and the chamber 108 can surround the membrane 106. Although the gaps 182, 188 have been described with respect to the cross-sectional view of FIG. 3, it is appreciated that other gaps between the peripheral end 190 of the membrane 106 and the substrates 102, 104, the chamber 108, etc., are similar to these gaps previously described for different cross-sections of the acoustic filter 100. As described in more detail below, the membrane 106 is advantageously free to move within the chamber 108 (e.g., side to side, such as perpendicular to the axial axis 180), and when a high pressure (or low pressure) acoustic wave is transmitted to the acoustic filter 100, the membrane 106 can block all the plurality of holes 148 (or the plurality of holes 128).

In some non-limiting examples, the chamber 108 can have a width 192, which can be defined between an inner surface of the substrate 102 (e.g., that is closest to the membrane 106) at the plurality of holes 128 and an inner surface of the substrate 104 (e.g., that is closest to the membrane 106) at the plurality of holes 148. In some cases, the inner surface of the substrates 102, 104 can be the portion of the respective substrate (e.g., a column) that is between two holes of that substrate. As shown in FIG. 3, the width 192 of the chamber 108 can be substantially uniform along the entire length of the chamber 108 (e.g., a longitudinal axis of the chamber 108 parallel to the axial axis 180 of the acoustic filter 100). Thus, in some cases, such as shown in FIG. 3, the chamber 108 can have a rectangular, square, etc., cross-section, and the chamber 108 can have a three-dimensional shape that corresponds to the cross-section (e.g., the chamber 108 as shown in FIG. 3 is a cylinder). In this way, with a substantially uniform cross-section along a longitudinal axis of the chamber 108, the membrane 106 (which can be flat) ensures flush abutment against each of the substrates 102, 104, which can ensure that the membrane 106 blocks a respective hole in the substrate 102, 104, and thus attenuates acoustic waves from passing therethrough. In some cases, a first portion of the chamber 108, a second portion of the chamber 108, and a third portion of the chamber 108 between the first portion and the second portion of the chamber 108, can have substantially the same width. In some cases, the first portion can be a first end of the chamber 108, the second portion can be a second end of the chamber 108 opposite the first end, and the third portion can be a center of the chamber 108.

In some non-limiting examples, the width 192 of the chamber 108 can define an amplitude threshold in which acoustic waves having an amplitude that exceeds (e.g., below or above) the amplitude threshold are attenuated by the acoustic filter 100, while acoustic waves having an amplitude that does not exceed the amplitude threshold (e.g., above or below) substantially pass through the filter 100. For example, when an acoustic wave (e.g., a sound wave) is applied to the acoustic filter 100 that has a large positive amplitude, the membrane 106 is forced against the substrate 104 (e.g., if the acoustic wave has a large positive amplitude or pressure), or against the substrate 102 (e.g., if the acoustic wave has a large negative amplitude or pressure, such as from an implosion) and covers the plurality of holes 148 of the substrate 104 (or the plurality of holes 128 of the substrate 102). In this way, when the membrane 106 abuts against a substrate and covers the holes of the respective substrate, acoustic waves (e.g., sounds waves) are blocked from passing therethrough, or in other words, are attenuated. Conversely, when an acoustic wave has a low amplitude, the membrane 106 is positioned away from the substrates 102, 104 (e.g., the membrane 106 does not cover the holes of either substrate) and the membrane 106 vibrates in response to the applied acoustic wave having a low amplitude so as to substantially allow the acoustic wave therethrough (e.g., the membrane 106 vibrates to effectively transmit the applied acoustic wave through the holes of a substrate). As the dimension of the chamber 108 that corresponds with the thickness of the membrane 106 (e.g., the width in FIG. 3) changes, so does the amplitude threshold. For example, as this dimension of the chamber 108 becomes larger, so does the amplitude threshold (e.g., only larger amplitudes are blocked or attenuated). Similarly, as this dimension of the chamber 108 becomes smaller, so does the amplitude threshold (e.g., smaller amplitudes are blocked or attenuated). Similarly, as the thickness of the membrane 106 increases, the amplitude threshold decreases (and vice versa). Accordingly, the membrane displacement along the dimension of the chamber 108 can dictate the amplitude threshold.

In some configurations, although not shown in FIG. 3, a dimension of the chamber 108 that corresponds to the thickness of the membrane 106 (e.g., the width 192 of the chamber 108) can be adjustable, which can thereby adjust the amplitude threshold. For example, the substrate 102 can be slideably engaged to the substrate 104 (or vice versa), such that as the substrate 102 is moved towards the substrate 104, the width 192 of the chamber 108 decreases and so does the amplitude threshold. Similarly, the substrate 102 can slide away from the substrate 104, such that the width 192 of the chamber 108 increases thereby increasing the amplitude threshold. In some cases, the acoustic filter 100 can include a lock, which can lock the position of the substrates 102, 104 (e.g., after a substrate has been slid relative to the other substrate) In this way, the amplitude threshold can be adjusted and locked to prevent the amplitude threshold from being changed (e.g., if the lock is engaged to lock the substrates 102, 104 together).

In some non-limiting examples, the width 192 (or other dimension of the chamber 108 corresponding with the thickness of the membrane 106) can be less than or equal to substantially 300 sim, 50 μm, 40 μm, 30 μm, 18 μm, 12 μm, 4 μm, etc. In some non-limiting examples, the acoustic filter 100 can have an amplitude threshold (e.g., a cut-off amplitude) that is less than or equal to substantially 160 dB, 155 dB, 150 dB, 140 dB, 115 dB, 95 dB, 85 dB, etc. In some cases, these amplitude thresholds can be advantageous in that these define cutoffs for which ear damage is likely to occur for various durations (e.g., with a 140 dB amplitude threshold, amplitudes exceeding the amplitude threshold will be attenuated so as to prevent a pain response from loud noises).

In some non-limiting examples, the amplitude threshold in dBs can be referenced relative to a conventional reference intensity value (e.g., a sound intensity value, such as 10⁻¹² W).

As shown in FIG. 3, each hole of the plurality of holes 128 can be aligned with a respective hole of the plurality of holes 148. For example, as shown in FIG. 3, the holes 122, 142 are aligned, the holes 124, 144 are aligned, the holes 126, 146 are aligned, etc. In some cases, each pair of aligned holes are not offset from each other (e.g., neither hole within a pair of holes extends above or below the other hole within the pair of holes and vice versa). In some non-limiting examples, each hole of the plurality of holes 128 can be fluidly coupled to the chamber 108 (and each recess 114, 120). Similarly, each hole of the plurality of holes 148 can be fluidly coupled to the chamber 108 (and each recess 114, 120). In some configurations, each substrate 102, 104 can include a recess, which can be directed into an opposing side of the respective substrate 102, 104 as the chamber 108. For example, the substrate 102 can include a recess 194 directed into the side 110 of the substrate 102 (e.g., opposite the side 112). As shown in FIG. 3, the recess 194 can decrease in cross-section in a direction towards the chamber 108, or stated another way, the recess 194 can increase in cross-section in a direction away from the chamber 108. In this way, sounds, including reflected sounds can be better directed (e.g., harvested) by the acoustic filter 100, such as sounds generated by the individual's voice that are reflected off objects and returned to the acoustic filter 100. Thus, the recess 194 can act similarly to a satellite dish, which can better “harvest” acoustic waves in the surrounding environment. In some configurations, a perimeter of the recess 194 can surround the plurality of holes 128, or in other words, each hole of the plurality of holes 128 can be aligned with the recess 194. As shown in FIG. 3, the recess 194 can be defined by a peripheral end 196 of the substrate 102, which can extend away from the chamber 108, and can partially or entirely extend around an axis of the substrate 102 (e.g., perpendicular to the axis 180). In some cases, the substrate 104 can include a similar recess as the recess 194 of the substrate 102, and thus the description of the recess 194 pertains to this recess. For example, the substrate 104 can include a recess 198 directed into the side 118 of the substrate 104 (e.g., opposite the side 116 of the substrate 104). A perimeter of the recess 120 can surround the plurality of holes 148, and the substrate 104 can include a peripheral end 200 that can extend away from the membrane 106 and can partially or entirely extend around an axis of the substrate 102 (e.g., perpendicular to the axis 180). In some cases, the recess 120 can have a uniform cross-section (e.g., as shown in FIG. 3), which can avoid harvesting unwanted acoustic waves (e.g., unlike the recess 194).

In some non-limiting examples, the membrane 106 can have a dimension (e.g., a length, a width, a diameter, etc.) such as other than the thickness of the membrane 106 that is smaller than a corresponding dimension of the chamber 108, the substrates 102, 104, the recesses 114, 120, etc. For example, as shown in FIG. 3, the diameter of the membrane 106 can be smaller than the diameter of the chamber 108, the substrates 102, 104, and the recesses 114, 120. In this way, the membrane 106 is advantageously free to move within the chamber 108 and avoids introducing undesirable resonant frequencies of the membrane 106, and attenuation losses at low acoustic amplitudes.

As shown in FIG. 3, an acoustic wave 202 (e.g., which can be a sound wave having a frequency within the hearing range of humans, such as less than 3 kHz), is directed at the acoustic filter 100. The acoustic wave 202 has an amplitude (e.g., an acoustic pressure) that exceeds (e.g., is larger than) the amplitude threshold of the acoustic filter 100. As the acoustic wave 202 propagates towards the membrane 106, since the amplitude exceeds the amplitude threshold, the membrane 106 is forced against the plurality of holes 148 of the substrate 104 thereby blocking the acoustic wave 202 from easily passing through the acoustic filter 100. This process significantly attenuates the acoustic wave 202, such that the resultant acoustic wave 204 that passes through the other side of the acoustic filter 100 is highly attenuated relative to the incoming acoustic wave 202. Conversely, an acoustic wave 206 (similar to the acoustic wave 202, such as being in the same frequency range) is directed at the acoustic filter 100 and has an amplitude (e.g., acoustic pressure) that is less than the amplitude threshold (e.g., the amplitude of the acoustic wave 206 is less than amplitude of the acoustic wave 202). As the acoustic wave 206 propagates towards the membrane 106, since the amplitude does not exceed the amplitude threshold (e.g., is below the amplitude threshold), the acoustic wave 206 causes the membrane 106 to vibrate freely (according to the acoustic wave 206), such that the emitted acoustic wave 208 remains largely unchanged. This is advantageous in that low amplitude sounds (e.g., at conversational volumes) are permitted through the acoustic filter 100, while high amplitude sounds (e.g., a blast) are attenuated by the acoustic filter 100. Advantageously, the acoustic filter 100 can automatically and nearly instantaneously change its configuration to respond to block high amplitude noises, and promptly resume transmission of low amplitude noises after the high amplitude noise ceases (and vice versa). This nearly simultaneous and instantaneous change in configuration advantageously avoids requiring complex electronic circuits including sensors, amplifiers, speakers, power sources (e.g., electronic power sources including a battery), which can be quite bulky and have lag time due to signal acquisition, processing, and output (e.g., to a speaker).

In some non-limiting examples, when an acoustic wave is below (e.g., does not exceed) the amplitude threshold of the acoustic filter 100, the acoustic wave can be transmitted through the acoustic filter 100 without substantially attenuating the acoustic wave (e.g., the inputted acoustic wave is attenuated less than 30% of the outputted acoustic wave, the acoustic wave is attenuated less than or equal to 20 dB, 10 dB, 5 dB, 3 dB, etc.). Stated another way, the amplitude of the acoustic wave at the input of the acoustic filter has about the same (or the exact same) amplitude as the amplitude of the acoustic wave at the output of the acoustic filter. In some non-limiting examples, when an acoustic wave is above (e.g., exceeds) the amplitude threshold, the acoustic wave can be attenuated by greater than or equal to 20 dB, 30 dB, or can be attenuated to a value between about 20 dB and 30 dB.

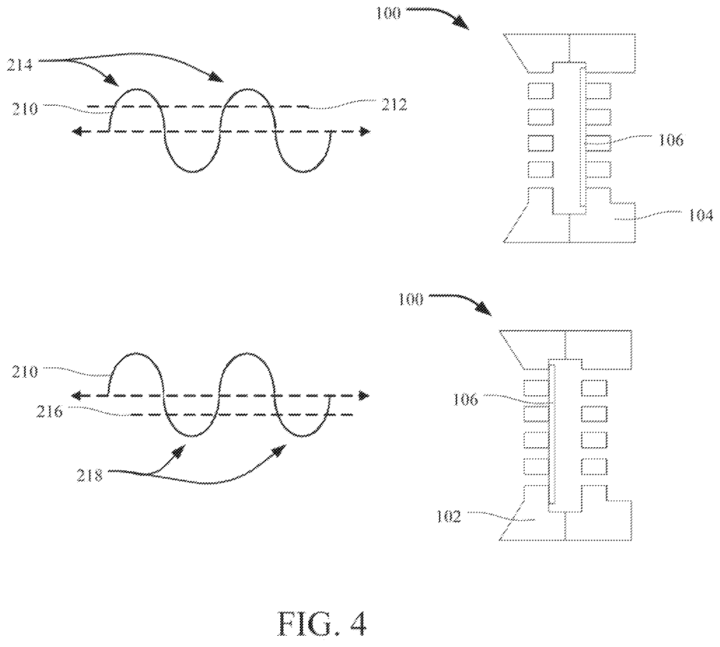

FIG. 4 shows the cross-sectional view of the acoustic filter 100 of FIG. 3 in two different attenuating configurations. The upper configuration of the acoustic filter 100 attenuates the acoustic wave 210 that propagates towards the acoustic filter 100. In particular, the portions of the acoustic wave 210 that are above the amplitude threshold 212 including the peaks 214 of the acoustic wave 210 are attenuated by the membrane 106 being forced against the substrate 104 and blocking the holes 148 thereby attenuating the acoustic wave 210, and in particular, the larger positive amplitude portions of the acoustic wave 210. Similarly, the lower configuration of the acoustic filter 100 attenuates the acoustic wave 210 that propagates towards the acoustic filter 100. In particular, the portions of the acoustic wave 210 that are below the amplitude threshold 216 (e.g., which can be the same as the amplitude threshold 212) including the peaks 218 of the acoustic wave 210 are attenuated by the membrane 106 being forced against the substrate 102 and blocking the holes 128 thereby attenuating the acoustic wave 220, and in particular the larger negative amplitude portions of the acoustic wave 210. In this way, the acoustic filter 100 can effectively attenuate both positive blasts (e.g., an explosion) and negative blasts (e.g., an implosion).

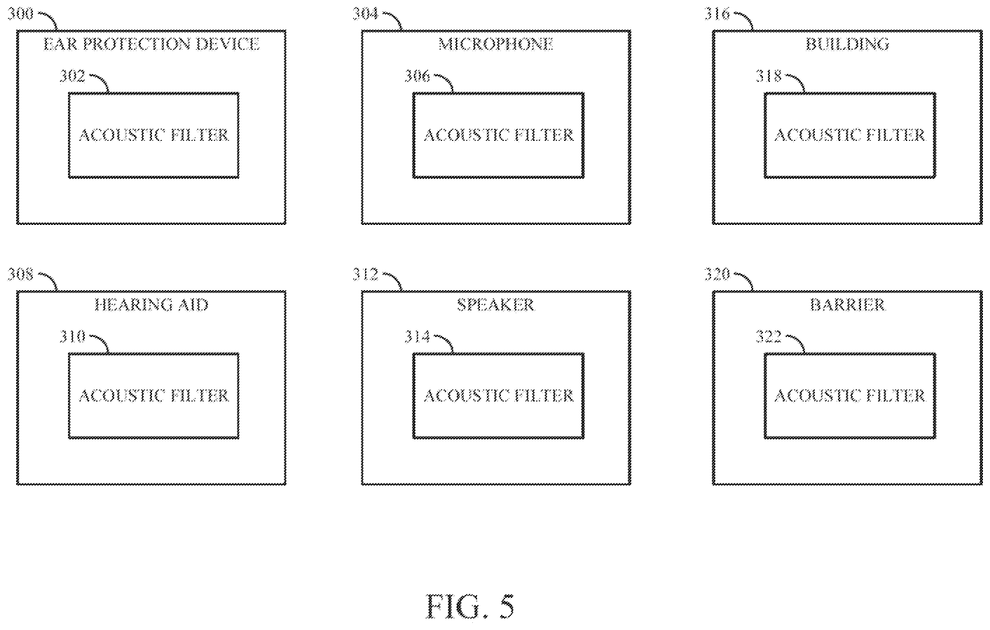

FIG. 5 shows a schematic illustration of various different devices, each of which can incorporate the acoustic filter 100 described previously. For example, FIG. 5 shows an ear protection device 300 (or in other words a hearing protection device 300) having an acoustic filter 302, a microphone 304 having an acoustic filter 306, and a hearing aid 308 having an acoustic filter 310. Although these schematic illustrations show these devices having one acoustic filter, in other configurations, each of these devices can have multiple acoustic filters. In addition, each of the acoustic filters can be implemented in a similar manner as any of the acoustic devices described herein (e.g., the acoustic filter). Thus, these acoustic filter pertain to the other acoustic filters described herein (and vice versa).

In some configurations, the ear protection device 300 can be an earplug, an earmuff, a helmet, etc. In some cases, the acoustic filter 302 can be positioned within the car protection device 300 (e.g., a body of the car protection device 300), coupled to the car protection device 300 (e.g., positioned exterior or interior to the ear protection device 300, such as at its body), etc. Regardless of the configuration, acoustic waves (e.g., sound) can be directed to pass first through the acoustic filter 302, and then pass into an ear of the user of the hearing protection device 300. The microphone 304 and the hearing aid 308 can be implemented in a similar manner as the ear protection device 300. For example, acoustic waves can be directed to pass first through the respective acoustic filter and then to the microphone (or other acoustic transducer). In this way, sounds that are too loud are avoided from being amplified, or sounds that are damaging to the microphone (e.g., a blasts) are avoided from damaging the microphone (e.g. the sensor of the microphone). In some configurations, the microphone can be part of a hearing device, such as a headset, headphone, headphones, ear bud, other devices facilitating hearing for a user (e.g., including a speaker). For example, in this case, a speaker 312 can be provided that can include an acoustic filter 314. In this way, sound from the speaker 312 (or ambient sound) can be directed first through the acoustic filter 314 and then can be directed to the user's ear. In this way, the user is protected from loud sounds from the speaker 312 or from the ambient environment that pass through the larger device that includes the speaker 312 (e.g., a headphone). In these configurations, an acoustic filter described herein can protect electronics from acoustic waves with large amplitudes (e.g., loud blasts).

In some configurations, the acoustic filter(s) can be implemented in other devices. For example, FIG. 5 shows a building 316 (e.g., a house, a stadium, etc.) including an acoustic filter 318. For example, a wall of the building 316 can include the acoustic filter 318 (e.g., the acoustic filter 318 coupled to or embedded within the wall), a window of the building 316 can include the acoustic filter 318, etc. In some cases, this can be advantageous for music instruction, concerts, sporting events, etc., that generate loud noises. Similarly, a barrier 320 (e.g., a fence, a highway median, other building structures, etc.) can include an acoustic filter 322. This can be advantageous in that, such as for a fence, loud sounds from neighbors can be significantly reduced. Similarly, this can be advantageous in traffic applications as medians incorporated with acoustic filters can reduce sounds resulting from other directions that may not be necessary in the current travel direction. Similarly to the other devices, although one acoustic filter 318 and one acoustic filter 322 is described, these devices, components, structures, etc., can have multiple acoustic filters (e.g., one, two, three, etc.).

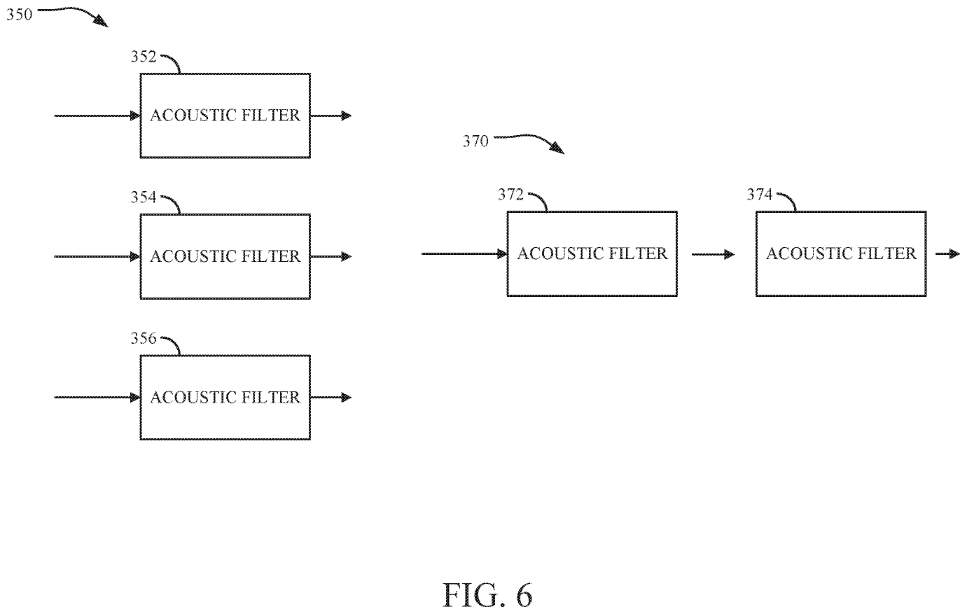

FIG. 6 shows a schematic illustration of the incorporation of multiple filters for devices. For example, FIG. 6 shows multiple acoustic filters 350, which can include acoustic filters 352, 354, 356. The multiple acoustic filters 350 are in a parallel (e.g., the filters 350 are not in series), which can be advantageous for areas that require larger surface areas. For example, as described below, multiple acoustic filters that are parallel can cover larger surface areas, such as those required for earmuffs. In this way, an acoustic wave (e.g., a sound wave) can be blocked by each acoustic filter 352, 354, 356, and the resultant acoustic wave can join together at the output (e.g., opposite from the input). In some cases, the amplitude threshold of the multiple acoustic filters 350 can be dictated by the acoustic filter of the multiple acoustic filters 350 having the highest amplitude threshold. In some cases, the amplitude threshold of each acoustic filter of the multiple acoustic filers 350 can be substantially the same.

FIG. 6 also shows multiple acoustic filters 370 that are in series (e.g., the filters 370 are not in parallel). The acoustic filters 370, which can include the acoustic filters 372, 374, being in series can be advantageous in that multiple acoustic filters can further attenuate loud acoustic waves (e.g., blasts) more so than a single acoustic filter. For example, an acoustic wave having a sufficiently high amplitude can first propagate through the acoustic filter 372 and generate a first attenuated acoustic wave. This first attenuated acoustic wave then propagate through the acoustic filter 374, which is further attenuated and generates a second attenuated acoustic wave, which is directed to the ear of a user (or other device, structure, sensor, etc.). In this case, each acoustic filter 372, 374 can have a similar amplitude threshold (e.g., the amplitude threshold of each acoustic filter 372, 374 is substantially the same), such that a given amplitude of an acoustic wave that exceeds both amplitude thresholds of the acoustic filters 372, 374 will be attenuated by both acoustic filters 372, 374. In other cases, if the acoustic filters 372, 374 have different amplitude thresholds, the amplitude threshold of the multiple acoustic filters 370 will be dictated by the acoustic filter of the acoustic filters 372, 374 having the lowest amplitude threshold.

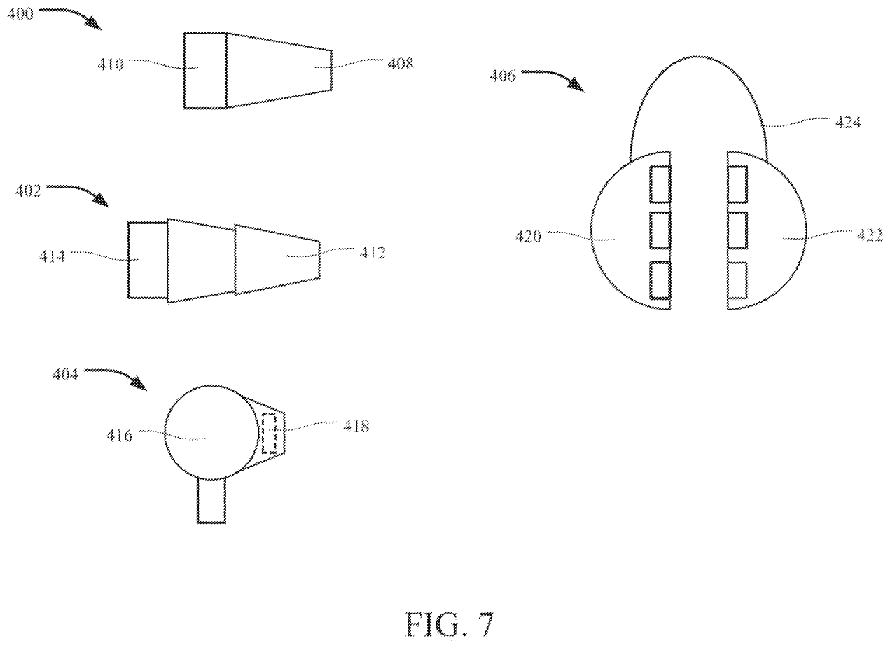

FIG. 7 shows schematic illustrations of specific implementations of the devices shown in FIG. 6. For example, FIG. 7 shows an example of earplugs 400, 402, a headphone 404, and earmuffs 406, each of which can include an acoustic filter (or multiple acoustic filters). The ear plug 400 can include a body 408 and an acoustic filter 410 coupled thereto or integrated within the body 408. For example, as shown in FIG. 7, the acoustic filter 410 can be coupled to an exterior side of the body 408 (e.g., further away from the ear canal than the interior side of the body 408), however in other configurations, the acoustic filter 410 can be coupled to an interior side of the body 408 (e.g., closer to the ear canal than the exterior side of the body 408). In some cases, the acoustic filter 410 being positioned closer to the ear canal can be advantageous in that the material of the body 408 can attenuate sound before the acoustic filter 410 attenuates the sound. In some configurations, the acoustic filter 410 can be configured to be positioned within an ear canal of the user. The ear plug 402 can be implemented in a similar manner as the ear plug 400. For example, the acoustic filter 414 can be coupled to or integrated within the body 412 of the earplug 402. In some cases, the body 412 of the earplug 402 can have different sized sections, each of which can have a smaller cross-section. In other words, the body 412 of the earplug 402 can taper until the body reaches 412 a distal end that is configured to be placed into an ear canal of the user, which is similar to the body 408, but with different distinct sections of the body 412.

The headphone 404 can include a body 416 and an acoustic filter 418 that is integrated within the body 416. The acoustic filter 418 can be positioned outside of the body 416 and can be coupled to the body 416. In some cases, the headphone 404 can include a speaker (e.g., within the body 416) and the acoustic filter 418 can be positioned in front of the speaker (e.g., so as to avoid allowing sounds that are too loud, generated from the acoustic filter 418, to pass into the ear of the individual). In other cases, the speaker of the headphone 404 can be positioned in front of the acoustic filter 418. In this way, the acoustic filter 418 avoids attenuating sounds from the speaker, but can attenuate sounds from the ambient environment. The earmuffs 406 can include ear coverings 420, 422, which can be coupled together with a headband 424. Each ear covering 420, 422 can include one or more acoustic filters. For example, as shown in FIG. 7, each ear covering includes multiple acoustic filters. Although the earmuffs 406, which can cover each ear, are described as being passive acoustic attenuators, in some cases, the earmuffs 406 can be headphones (e.g., over the ear headphones).

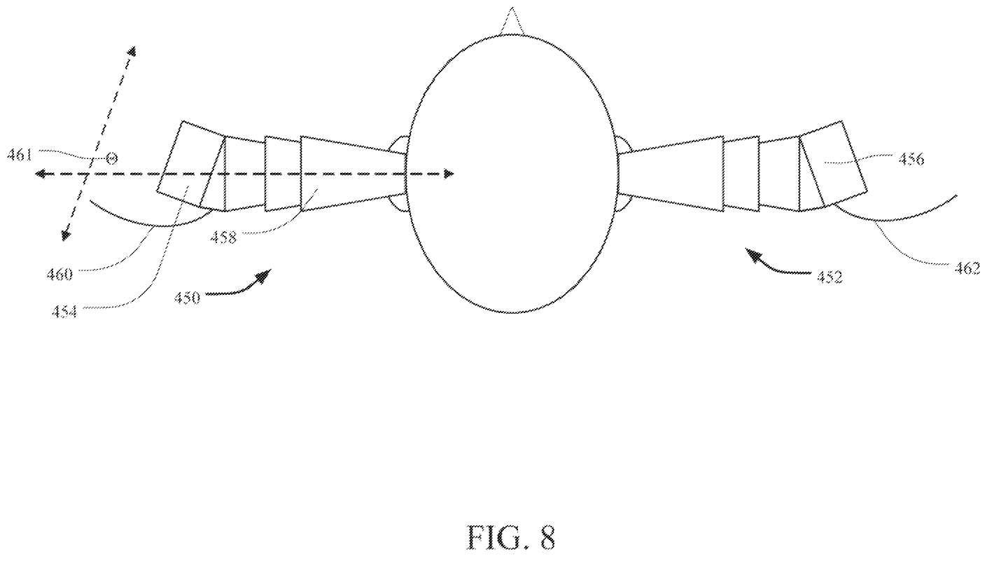

FIG. 8 shows an example illustration of earplugs 450, 452 that are enlarged relative to the individual to highlight features of the earplugs 450,452. Each earplug 450, 452 is shown as being positioned within a respective ear canal of the subject. In some non-limiting examples, each ear plug 450, 452 can include a respective acoustic filter 454, 456, which is shown as being angled relative to a longitudinal axis of the representative body of the ear plug that it is coupled thereto or integrated within. For example, the acoustic filter 454, and more specifically a housing of the acoustic filter, is angled relative to the longitudinal axis of the body 458 of the ear plug 450. In this way, the acoustic filter 454 (and the membrane of the acoustic filter 454) are angled relative to the ear canal of the subject (e.g., the ear canal in which the earplug 450 is positioned in). Accordingly, sounds that reflect off objects including the voice of the individuals wearing the earplugs 450, 452 are better collected by and directed to the acoustic filter 454. In this way, individuals are better able to hear their own voice when ambient sound amplitudes are relatively low. In some cases, the angle 461 of the acoustic filter 454 relative to the longitudinal axis of the body 458 (or the longitudinal axis of the ear canal, such as at the entry of the ear canal) can be less than or equal to substantially 80 degrees, 70 degrees, 60 degrees, 50 degrees, 40 degrees, 30 degrees, 20 degrees, 10 degrees, etc. As the angle 461 becomes smaller, the acoustic filter 454 is better able to harvest reflected sounds, but with the earplug 450 becoming larger (e.g., having a larger spatial footprint). In a similar regard, each earplug 450, 452 can include an acoustic reflector 460, 462. The acoustic reflector 460, which can be coupled to the body 458, can harvest sounds from the ambient environment, especially those that reflect off objects (e.g., the individual's voice). The acoustic reflector 462 and the angling of the acoustic filter 456 can function as a similar manner as the acoustic filter 454 and the acoustic reflector 460. While the angled acoustic filter and the acoustic reflectors are demonstrated with respect to ear plugs, it is appreciated that these features can be applied to other devices described herein (e.g., earmuffs).

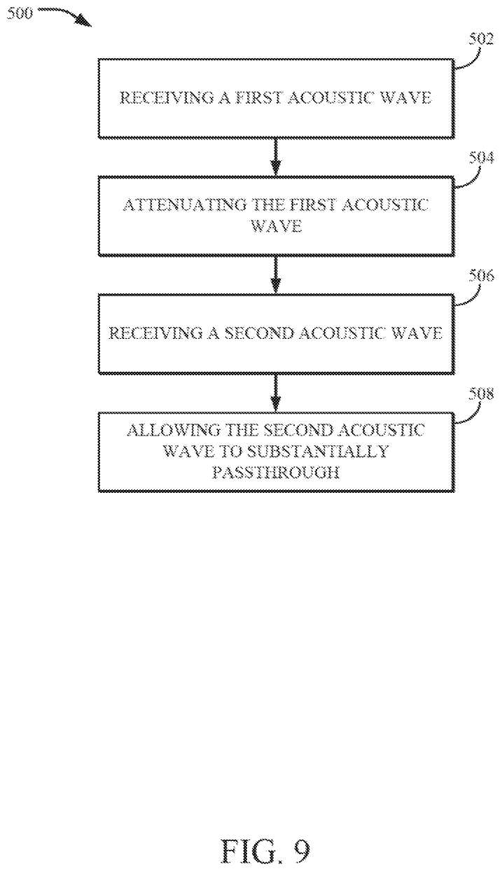

FIG. 9 shows a flowchart of a process 500 for selectively attenuating acoustic waves, which can be implemented using any of the devices described herein, such as, for example, the acoustic filter 100. At 502, the process 500 can include receiving a first acoustic wave (e.g., with an acoustic filter). For example, the first acoustic wave can be directed at an acoustic filter, and the acoustic filter can receive the first acoustic wave. In some cases, the first acoustic wave can have a first amplitude (e.g., a pressure) that exceeds (e.g., is greater than) an amplitude threshold of the acoustic filter. In some configurations, the first acoustic wave is a blast, an implosion, an explosion, other loud sound impulse, etc.

At 504, the process 500 can include attenuating the first acoustic wave. In some cases, this can include the acoustic filter, in a first configuration (e.g., a first operational configuration, an attenuating configuration, etc.), attenuating the first acoustic wave (e.g., as the first acoustic wave passes through the acoustic filter). In some non-limiting examples, this can include the first acoustic wave forcing a membrane of the acoustic filter against a substrate that includes multiple holes. In some cases, this can include the membrane covering each hole of the multiple holes to attenuate the first acoustic wave. In some cases, attenuating the first acoustic wave can attenuate the first acoustic wave by a particular value, such as, for example, greater than or equal to substantially 20 dB, 30 dB, between substantial 20 dB and 30 dB, etc.

At 506, the process 500 can include receiving a second acoustic wave (e.g., with the acoustic filter). For example, the second acoustic wave can be directed at the acoustic filter, and the acoustic filter can receive the second acoustic wave. In some cases, the second acoustic wave can have a second amplitude (e.g., a pressure) that does not exceed (e.g., is less than) an amplitude threshold of the acoustic filter. In some cases, the second acoustic wave can be ambient noises, conversational sounds (e.g., from humans), etc.

At 508, the process 500 can include allowing the second acoustic wave to substantially passthrough (e.g., the acoustic filter). In some cases, this can include the acoustic filter, in a second configuration (e.g., a second operational configuration, a non-attenuating configuration), allowing the second acoustic wave to substantially passthrough the acoustic filter without substantially attenuating the second acoustic wave (e.g., where the attenuation of the second acoustic wave is less than the attenuation of the first acoustic wave, less than or equal to 30 dB, less than or equal to 20 dB, less than or equal to 10 dB, less than or equal to 5 dB, less than or equal to 5 dB, etc.). In some non-limiting examples, this can include the second acoustic wave causing the membrane of the acoustic filter to vibrate freely (e.g., within the chamber) thereby transmitting the second acoustic wave through the acoustic filter, with the membrane being positioned away from the first substrate and the second substrate (e.g., the membrane not in contact with the first substrate or the second substrate, the membrane not covering holes of the first substrate or the second substrate, etc.).

In some non-limiting examples, depending on the amplitude of the incoming acoustic wave, the acoustic filter can nearly instantaneously switch between the first configuration (e.g., attenuating configuration) and the second configuration (e.g., the non-attenuation configuration) to condition (or not condition) the incoming acoustic wave. Thus, although the process 500 proceeds in a particular direction, the blocks of the process 500 can be implemented in different orders, as appropriate. In addition, with the nearly instantaneous switching between the two configurations, the acoustic filter allows for a passive non-linear attenuation over different amplitudes. Accordingly, the acoustic filter advantageously does not require electronics (e.g., amplifiers, analog-to-digital converters, power sources, etc.) to selectively condition incoming acoustic waves depending on the amplitude of the acoustic wave.

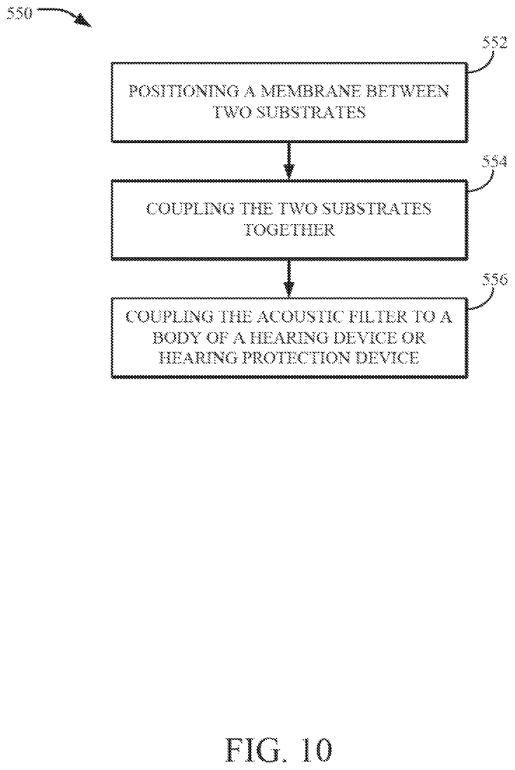

FIG. 10 shows a flowchart of a process 550 for manufacturing an acoustic filter and can include a method of manufacturing a hearing protection device or other devices described herein (e.g., a headphone). At 552, the process 550 can include positioning a membrane between two substrates. In some cases, this can include positioning a membrane within a chamber defined by the two substrates.

At 554, the process 550 can include a coupling the two substrates together (e.g., with the membrane positioned within the chamber). In some cases, this can include coupling respective peripheral ends of the substrates together (e.g., using an adhesive, such as a superglue). In some cases, and advantageously, this can include avoiding coupling the membrane to either substrate. For example, a dimension of the membrane other than a thickness of the membrane (e.g., a width, a length, a diameter, etc.) can be smaller than a corresponding dimension of the chamber, and each substrate. In this way, the membrane is free to move within the chamber so as to prevent introducing unwanted attenuation at low sound amplitudes and introducing undesirable resonant frequencies attributable to the membrane. In some cases, with the membrane positioned within the chamber, the membrane is not taught, is not tensilely loaded, etc., which avoids introducing the resonant frequencies and introducing unwanted attenuation at low sound amplitudes. In some cases, this can include positioning the membrane entirely within the chamber. This can also include sealing the membrane within the chamber (e.g., hermetically sealing the membrane within the chamber).

At 556, the process 550 can include coupling the acoustic filter to a body of a hearing device or a hearing protection device. In some cases, this can include positioning the acoustic filter within the body, or embedding the acoustic filter within the body (e.g., to protect the acoustic filter from damage (e.g., from dropping the device).

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

### Example 1

The core technology disclosed comprises a nonlinear filter. The filter may be comprised of a thin membrane sandwiched between a pair of stopper grids. At low SPL, the membrane vibrates freely transmitting sound with low loss. In the event of blast, the large displacement of the membrane is constrained by the stopper and the transmitted maximum pressure is limited. When the blast is gone, the membrane goes back to the normal free vibration. As a result, the transmitted SPL has a nonlinear function with a cutoff SPL adjustable by controlling the gap between the stopper grids.

This disclosure relates to methods and apparatuses for providing nonlinear attenuation of sound to protect the ear against loud sounds and blasts, such that it provides high attenuation for high sound pressure level (“SPL”) sound but does not affect the hearing of normal SPL sound. The present disclosure relates generally to devices for controlling acoustic wave transmission. A primary application of such devices is hearing protection in the form of earplugs, an earmuff, or a headset, to protect the user from ear injuries or hearing loss by loud noise or blast noise.

Auditory injuries are among the most common primary injuries resulting from improvised explosive devices, rocket propelled grenades, and mortar rounds. Many U.S. soldiers have sustained blast related injuries in the operation against Iraq in 2003. Normal human eardrums rupture at a sound pressure level (“SPL”) of 190-200 dB. When the eardrums are perforated, one's hearing ability is greatly compromised. Moreover, high intensity sound above 120 dB SPL, when transmitted to the inner ear, can cause damages to hair cells in the cochlea. Hearing loss is the second most common disability among the U.S. forces. Use of hearing protection devices, such as foam earplugs and other “combat” earplugs with caps, can reduce the risk of hearing loss, but only in the case when the soldiers are alerted and have time to put on the protection devices before loud sounds. Active earplugs or earphones with electronic intensity filters can also protect soldiers, but they do not allow the soldiers to hear natural sounds transmitted in the air, which can limit their ability to recognize and localize sounds.

Normal human eardrums rupture at a sound pressure level (SPL) of 190-200 dB. Once the eardrum is perforated, the subject's hearing ability is greatly compromised because sound coupling to ossicles is disrupted. Moreover, high intensity sound transmitted to the inner ear can cause damages to hair cells in the cochlea. According to year 2010 data from the Hearing Center of Excellence, Department of Defense, hearing loss is the second most common disability among the U.S. forces. Military personnel as well as civilians can experience substantial blast-induced injuries to the ear from improvised explosive devices, rocket propelled grenades, mortar rounds, etc. The most common injury is the rupture of the eardrum, which typically occurs at sound pressure levels (SPL's) above 160 dB (i.e., greater than 2,000 Pa). High intensity sound conducted through an intact eardrum to the inner ear may cause acute and chronic damages to the hair cells in the cochlea. Identified by the Department of Veteran's Affairs as the second most common new disability among the U.S. forces, not only is the hearing loss a tactical risk that threatens combat effectiveness, but it also deteriorates the future living quality of Soldiers and causes a significant financial cost.

Use of hearing protection devices, such as foam earplugs and other “combat” earplugs with caps, may reduce the risk of hearing loss. However, passive damping earplugs tend to decrease the ability to determine the direction of a sound source and to hear common verbal communications. When manual caps are open, the users can hear natural sounds. Many hearing protection devices including Moldex BattlePlug™ and 3M™ Combat Arms Earplug™ rely on a manual cap. However, the manual adjustment reduces the effectiveness especially due to the unpredicted nature of blasts in the field.

Passive earplugs with nonlinear transmission filters, such as orifice filters and sound-induced movable plugs, have shown to provide some, but insufficient, level of protection and tend to suffer from unsatisfactory hearing qualities due to relatively large transmission loss and phase distortion leading to reduced awareness and directionality. Active electronic hearing protection devices also exist, providing dynamic range compression. However, these active devices require built-in power sources, are expensive, and do not reproduce all the qualities of natural sound waves. The inability to hear natural sounds may limit their ability to recognize and localize sounds.

There are also several patents describing design concepts for nonlinear transmission acoustic devices. In U.S. Pat. No. 8,249,285 (“Method and Apparatus for Producing Nonlinear Sound Attenuation”; incorporated herein by reference in its entirety), the inventors proposed using a flexible diaphragm on a perforated disk with the holes of any size and shape. The diaphragm expands upon an increase in the external sound pressure level. The diaphragm can be made of extremely thin materials like polyethylene or Teflon foil. In the example materials, the inventors failed to consider elastic materials. The deflection of a flexible (but not elastic) diaphragm under a few kPa pressure difference is very small, and the design may require very high air pressure that may not be feasible with the typical blast pressure values. More importantly, the diaphragm in this design is connected and held from the edges. An important part of our disclosure is that the membrane is not held at all. Rather, it is freely placed inside a small gap without connecting, gluing, holding or anything that can constrain it and produce acoustic loss and membrane resonances. The free membrane design disclosed in this application, provides very low transmission loss for low pressure input sound and provides normal hearing sense. This is a goal of the design and the device may not be able to realize these characteristics if the membrane is not free.

US patent publication 2017/0200440 (incorporated herein by reference in its entirety) suggests using a movable diaphragm supported by springs that anchors to the substrate, or a movable diaphragm wherein the diaphragm has at least one hole on it. Like the other device in U.S. Pat. No. 8,249,285, the diaphragm in this design is held and constrained, and thus is different from the free membrane design described in this application. Moreover, this device only provides one-way protection. In the design disclosed herein, the membrane is placed between a pair of stopper grids and therefore functions for both positive- and negative-pressure parts of a blast.

The present disclosure provides systems and methods for hearing protection devices or earplugs to protect a user from sound-induced ear injuries including blast-induced hearing losses.

Some non-limiting examples of the disclosure are described herein with reference to the accompanying Figures. The description, together with the figures, makes apparent to a person having ordinary skill in the art how some non-limiting examples of the disclosure may be practiced. The figures are for the purpose of illustrative discussion and no attempt is made to show structural details of an non-limiting example in more detail than is necessary for a fundamental understanding of the teachings of the disclosure.

The disclosure will be explained with the following order:

### Filter Design and Parts

The filter consists of a thin membrane with low transmission loss sandwiched between a pair of low-loss stopper grids. There is a small spacing between the grids' inner surfaces which is called the “filter gap”. The membrane is placed inside the gap without any bond or gluing of the membrane edges to the grid.

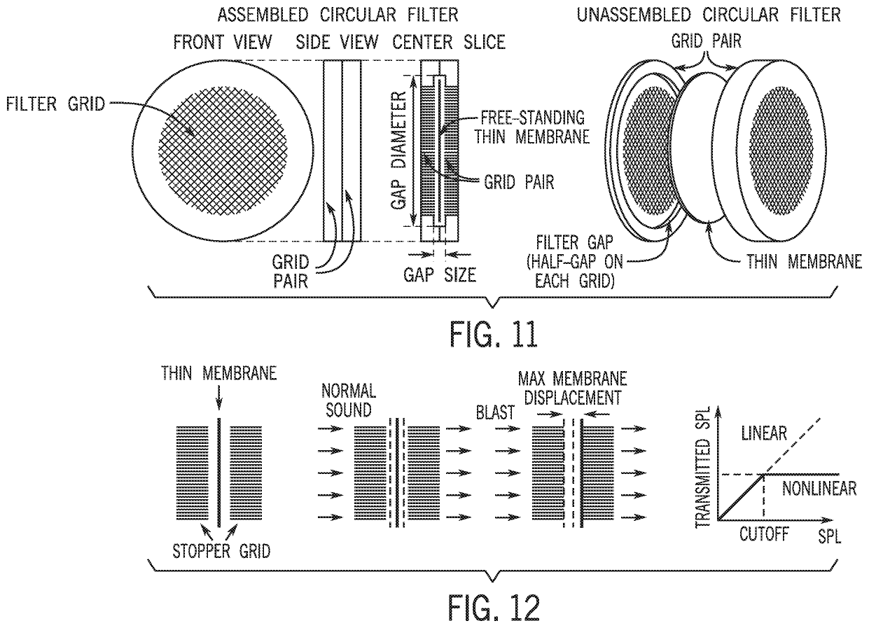

FIG. 11 shows a front view and a side view of a grid of the filter, along with a cross-section of an assembled acoustic filter as well as an exploded view of an assembled acoustic filter.

FIG. 12 shows schematic illustrations of the membrane of the acoustic filter in different positions and a graph illustrating the non-linear attenuation profile of the acoustic filter.

### Filter Working Mechanism

Upon normal SPL input the amplitude of the membrane vibrations is smaller than the small grid size. Consequently, the membrane freely vibrates inside the filter gap and the sound transmits through the filter with minimal attenuation. The membrane and grids are designed to have minimal transmission loss over a desired acoustic spectrum for normal SPL input. This shows the filter ON state. In response to an incoming blast above a threshold, the membrane displacement exceeds the gap size. Consequently, the stopper grids constrains the membrane displacement, and the filter is switched to the OFF state with low acoustic transmission. The OFF state prevents the blast from being transmitted through the filter. The SPL threshold level of the filter is designed by the filter gap size and can be modified based on the application. The switching mechanism is automatic, instant and reversible; and after blasts, it goes back to the ON state, allowing for normal hearing.

### Grid Holes Size

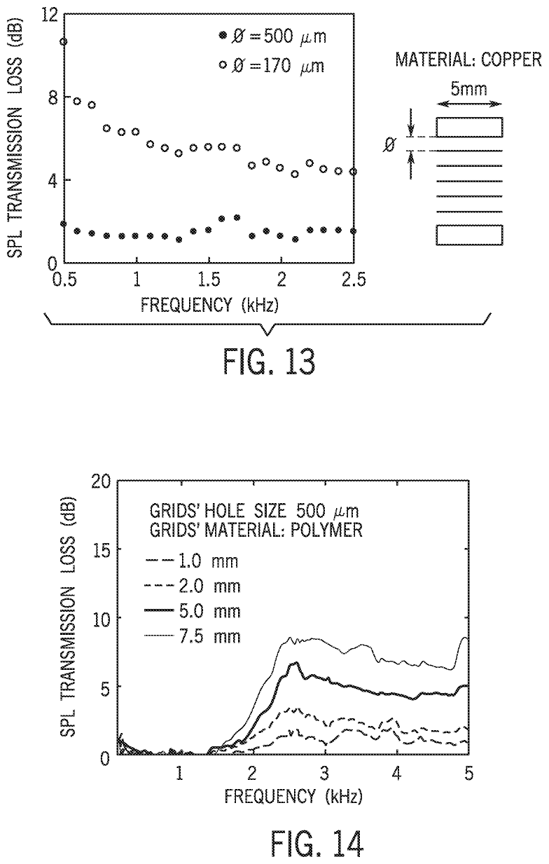

The holes size of the grids cannot be too small and too large. A grid with large holes will not stop the whole membrane surface deflection. Reducing the size of the grid holes, increases the transmission loss for lower frequencies due to viscous damping. To verify this hypothesis two grids were made from copper. Metals have low acoustic absorption, so the sound attenuation of these grids is mainly due to the geometrical structure rather than the material properties. The filling factors of the grids with 500 μm and 170 μm are 0.31 and 0.14, respectively and their thickness was 5 mm. The sound attenuation for the small-hole grid increases dramatically for the lower frequencies (See FIG. 13).

FIG. 13 shows a graph of the attenuation of different acoustic filters having different sized holes.

A desirable grid should have the smallest possible hole size that it does not cause any sound attenuation. The optimum size of the holes also depends on the grid thickness, 500 μm hole size is an acceptable size for a 5 mm thick grid. The hole size can be smaller for a 1 mm thick grid.

### Grid Thickness

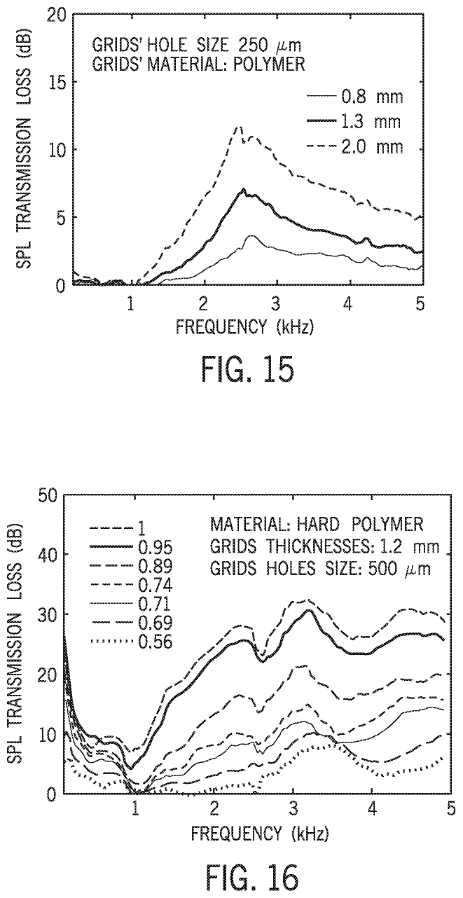

The grid is good to be as thin but mechanically strong. Thick grids may enhance the transmission loss via material absorption. Viscous damping and high frequency acoustic resonances also get intensified through longer and narrower grid holes. Each line of the plots in the FIG. 4 show the transmission loss for a single grid of a specific thickness. The grids in this test are made from a polymer, which unlike the metallic grid, is absorptive in some frequencies. This polymer material has higher absorption at 2-3 kHz than copper. This test shows that thicker grids have higher transmission loss. Grids with smaller holes size (See FIG. 15) show higher transmission loss than the large hole grids of the same length (See FIG. 14).

FIG. 14 shows a graph of the frequency response for grids of different thicknesses.

FIG. 15 shows a graph of the frequency response for grids of different thicknesses.

For a grid with 500 μm hole size, 1 mm thickness or less is good. Thinner grids are advantageous if they are made from a material that provides enough strength. Thinner thickness of the grid allows making the holes size to be smaller.

### Grid Filling Factor

Filling factor (ft) is defined as the ratio of a grid surface area after subtracting the holes area to the whole grid surface.

$$FF = 1 - \frac{holes area}{total area} .$$

So, FF=1 and FF=0 represent “fully blocking grid” and “no grid” cases, respectively. According to the tests results that are summarized in the FIG. 16, it is important that the ff of the stopper grid be as small as possible to provide low loss for low-SPL input sound. To obtain a small ff value, the grid inner wall must be as thin as possible.

FIG. 16 shows a graph of the frequency response for grids having different filling factors.

### Grid Material

It can be important that the Grid be made from a rigid material that does not deform or flex when using the filters and the small gap size remains fixed. Any change in the gap size can affect the filter nonlinear threshold and the functionality of the device. The grid material should also be mechanically strong enough that the thin inner grid walls remain stable and fixed. The damaged walls can affect the filter performance. The exact mechanical parameters of the grid depends on the grid dimensions. A wide range of materials with different fabrication techniques can be used for fabricating the grids, including stacking metallic or polymeric capillary tubes, extruding and thermal drawing of thermoplastic polymer preforms, 3D-printing of polymeric resins, etc.

### Grid Holes Shape

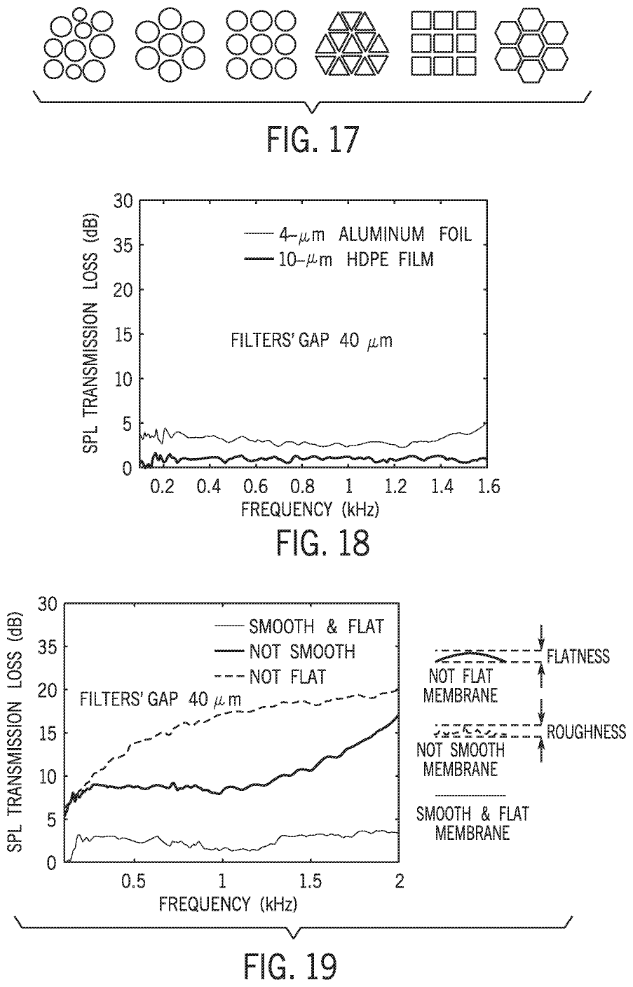

The grid holes can be of any shape, such as triangular, circular, square, hexagonal, etc. and can be at any relative position respect to each other (FIG. 17). However, to obtain a small FF value, they should be dense and take up as much area of the grid surface as possible. They can randomly or orderly positioned. They can be of the same or different sizes; however, a very small or very large hole can negatively affect the performance of the filter.

FIG. 17 shows a schematic illustration of different shapes for the holes of the grids.

### Grid Shape

The filter and the grid can be of any shape and size. It is important that the grid surface be flat, and the gap size does not change all over the grid surface. A hearing protection device can have several filters or one filter with a customized shape. It can be small to be used as an earplug or can be large to cover an earmuff surface.

### Membrane Material

Each filter has a thin membrane. The membrane can be made from a wide range of materials, including flexible and rigid material. It can be made from polymers, thin metallic foils, or any other material than can be in the form of thin films and can fully block the air flow. FIG. 18 shows the SPL transmission loss of the two filters with metallic and polymer thin membranes for conversational level input sound. Both filters show very good transmission for low-SPL input sound. If the membrane is made from a very soft material, it may get deformed inside the gap because of its own weight and the low mechanical rigidity. This has negative impact on the filter functionality. The membrane should be made from a material that can withstand its own weight. Unlike other devices, such as the one in U.S. Pat. No. 8,249,285, the membrane as described herein should not be glued or attached on the edges to the grids. Bonding the edges of a rigid membrane increases the transmission loss of the filter in the ON state. Bonding of a soft and elastic membrane results in appearing acoustic resonances and increases the transmission loss. Furthermore, a soft and elastic thin film can undergo local deformation on each single hole of the grid in presence of a high-SPL blast and be less successful in blocking it.

FIG. 18 shows a frequency response for membranes having different thicknesses and materials.

### Membrane Apparent Shape

It is better if the filter membrane is flat and smooth. The surface roughness, small wrinkles and tiny folds should be in the order of the filter gap or smaller. The membranes may be pressed to become smooth. Pressing a membrane may cause it to curve. The large membrane roughness or the surface curvature that makes the membrane to be continuously in touch with both grids can stop the membrane to freely vibrate inside the gap and increase the transmission loss of the filter in the ON state. FIG. 19 depicts the importance of smoothness and flatness of the membrane. The three filters have a 10 μm high density polyethylene (“HDPE”) membrane placed inside a 40 μm gap. The filter with a flat and smooth membrane has low SLP transmission loss in the ON state, while the other two without smooth or flat membranes have high transmission losses. Membranes should also be free of electric charges that can potentially attract them to the grids and constrain the vibrations.

FIG. 19 shows a graph of the frequency response for membranes having different properties.

### Membrane Thickness

The membrane should be thin but mechanically strong. It should be thinner than the filter gap to be able to freely vibrate in response to the incoming low-SPL sound. A thinner membrane has less mass and with less inertia can respond quicker and stronger to the incoming sound and transmit it with less distortion. The exact thickness of the membrane depends on the material density, material mechanical strength, and the gap size. A membrane made of a high-density material such as a metallic foil should be thinner than a polymeric membrane to have the same performance. To have a filter with a lower SPL threshold the gap should be smaller. The membrane should be thin enough to fit in a small gap. On the other hand, a very thin membrane may not be strong enough to survive the blasts and possible mechanical tensions. As an example, an ideal thickness for a membrane made of HDPE polymer with 1 cm diameter inside a 30 μm gap size, can be less than 20 μm.

### Membrane Size

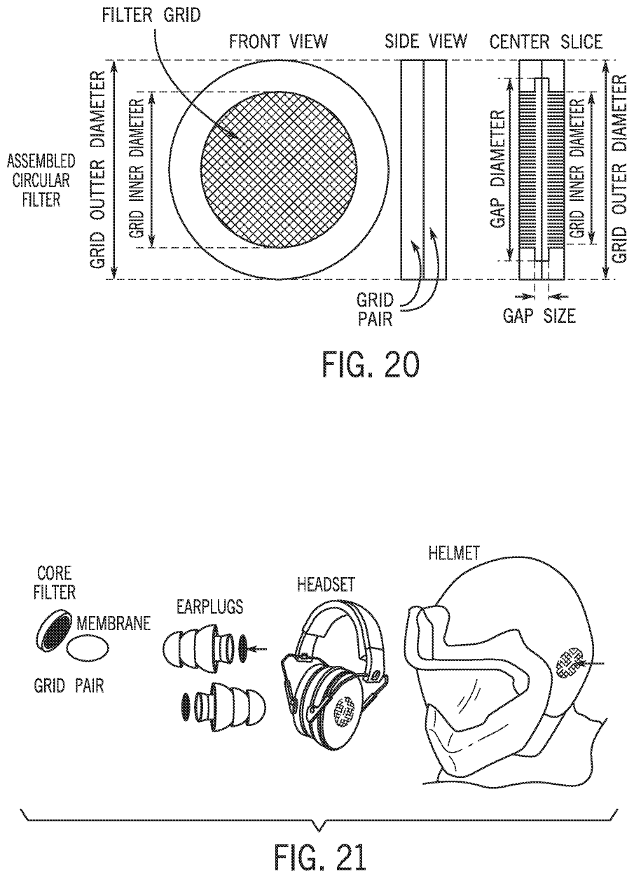

For any customized shape of the filter and the grids, the membrane should be big enough to cover all the grid holes, but not too big that does not fit in the gap. A smaller membrane cannot fully block a blast. For a circular filter like the one in FIG. 20, the membrane diameter should be: grid diameter+(gap diameter−grid diameter)/2<membrane diameter<gap diameter.

FIG. 20 shows a schematic illustration of a front view of a grid of an assembled acoustic filter, a side view of the assembled filter, and a cross-sectional view of the assembled filter.

### Gap Size

The size of the gap defines the SPL threshold level of the filter. A filter with too large a gap cannot constrain the large displacement amplitude of the membrane in presence of a blast and consequently does not provide enough transmission loss. On the other hand, the gap cannot be too small that freezes any vibration of the membrane which results in high loss in the ON state of the filter. According to the tests herein, a filter with a 10-μm-thick and 1-cm-diameter HDPE membrane, and a 30 μm gap provides ˜40 dB loss for a blast with 160 dB SPL peak. This amount of loss can bring the transmitted SPL bellow the ears pain threshold. A slightly smaller gap can enhance the loss, however there can still be sound transmission through the other parts of the earmuff or through the skull.

Hearing Protection Devices with Nonlinear Filters

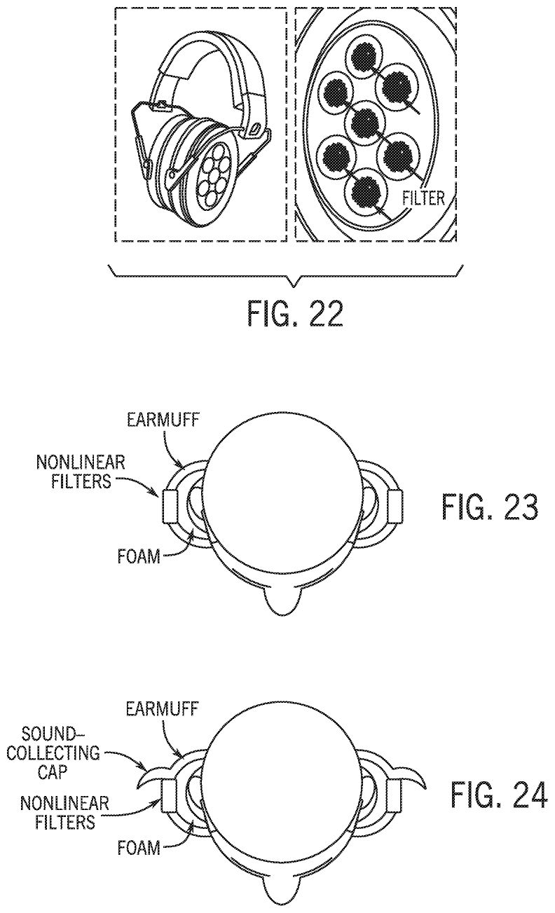

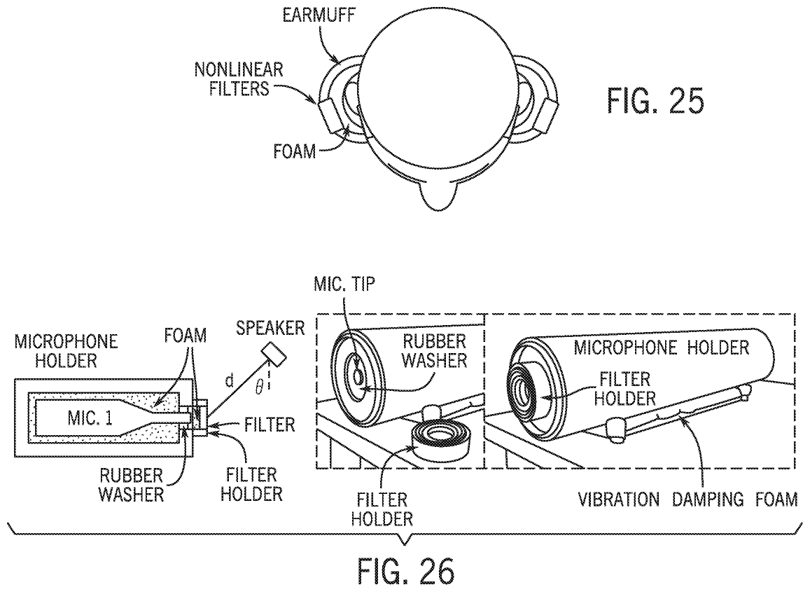

The filter can be integrated in a commercially available device such as an earmuff or a helmet. It can also be miniaturized to be used in an earplug. The earmuff, earplugs, and helmets can be a commercially available hearing protection product, but after modification and mounting the filters, it can provide nonlinear sound transmission. The filter enables the warfighters to hear natural sounds below a set threshold while being compatible with built-in wireless human interface devices. FIG. 21 shows various hearing protection devices employing nonlinear filters (arrows). Small filters can be used to make nonlinear earplugs. There can be one pair or more filters on each device of earmuffs and helmets. FIG. 22 shows an earmuff prototype, upgrading an earmuff with the filters described herein. There are 7 filters on each side of the earmuff. The filters can be of any customized shape and size. The spacing between the backside of the filter or filters and the ear can be partially filled with soft foams to prevent acoustic resonances and minimize the sound distortion (FIG. 23). One reason for an anormal sense of hearing after wearing the hearing protection devices is the way that the person hears his/her own voice. When the ears are covered with a protective device, the sound of speech is mainly heard internally through the middle ear. But without the protective devices we can hear our own voice externally after reflections from the objects. The nonlinear filters are designed to have minimal acoustic attenuation and distortion on the incoming sound in the ON state. However, to obtain a normal sense of hearing there should be a balance between the internal and external hearing contributions of our own voice. A tilted or curved cap on the filters can improve the contribution of external hearing of our own voice (FIG. 24). The cap can be made from any material such as metals, polymers, foams or other materials than can reflect the sound to the filters. Another possible configuration is the tilted-filters, in which the filters are orientated to the front side (FIG. 25) The tilting helps to get more of the reflected sound from the front side through the filters.

FIG. 21 shows schematic illustration of various hearing protection devices that can include an acoustic filter.

FIG. 22 shows an illustration of an earmuff prototype with multiple acoustic filters distributed within an ear cover.

FIG. 23 shows a schematic illustration of a top view of an earmuff including multiple acoustic filters.

FIG. 24 shows a schematic illustration of a top view of another earmuff including multiple acoustic filters and a tilted or curved cap that can function as an acoustic reflector.

FIG. 25 shows a schematic illustration of a top view of another earmuff including multiple acoustic filters that are tilted relative to the user.

### Experimental Setup for Low-SPL Transmission Test

To measure the filters transmission loss for low-SPL input sound, an experimental test setup was built (FIG. 26). In this test, a microphone inside a metallic holder could measure and record the sound produced by a speaker. The speaker can be a commercially available product. A micro-speaker of a cellphone was used. The test sound is a 90-dB-loud brown noise or while noise, which have all the audible frequencies. The filter is placed inside a metallic holder which is connected to the microphone filter using threads. The microphone holder has a hard metallic body and is placed on some foam to damp the vibrations in the environment. The microphone inside the holder is covered with some foam. The foam operates to fill all the empty spaces and eliminate the formation of acoustic resonances inside the holder. They also damp the undesired vibrations from the microphone holder. The front side of the microphone is held by a rubber washer. It keeps the microphone tip at a fixed position during the tests, and reduces the sound entering the microphone holder and forming undesired resonances. For the same purpose, the gap between the filter and microphone tip is filled with the damping foam. The micro-speaker is placed at 6 cm from the filter and at an angle of 45°, as shown in FIG. 26.

FIG. 26 shows an experimental set-up for testing the acoustic filter.

The 6 cm distance allows replacing the filters throughout the test without touching the microphone. It should not be too far as well. Because there are acoustic resonances inside the lab due to the reflections from the walls. If the speaker is close to the microphone, the intensity of the directly incoming sound is much louder than the reflections and the formed resonances. If the tests are performed inside an acoustic room with perfectly absorbing walls, this distance can be larger. The angle of the speaker-filter direction with respect to the filter facing direction can be smaller or larger. Placing the speaker right in front of the filter can form unwanted resonances. If the angle is too small, the sound will not efficiently hit and enter the filter. The microphone holder, filter holder and the dimensions are designed to have the minimal distortion on the incoming sound. This was verified by measuring the spectral contents of a brown noise, once when the microphone and speaker were placed in the test setup, and once without the setup and by placing the speaker in front of the microphone. The difference between the two spectra was less than 3 dB over the conversational spectral range (0-3 kHz). The setup was also tested for the possible sound leaks. The spectral transmission over the audible spectral range drops by 30-40 dB if the filter is replaced with a blocking disk of the same material and thickness. To measure the filter transmission, the microphone records the white noise of the speaker sounds for 30 seconds in two cases. In the first recording (so called “Sig”) the filter is placed inside the filter holder, and in the second recording (so called “Ref”) the filter is replaced with a ring. The ring is made from the same material as the filter and has the same thickness and outer diameter. The inner diameter of the ring is the same as the grid inner-diameter of the filter (FIG. 20). The recordings are plotted in frequency domain and in dB scale.

$$Transmission is defined as : T = Ref ( dB ) - Sig ( dB )$$

### Experimental Setup for High-SPL Transmission Test

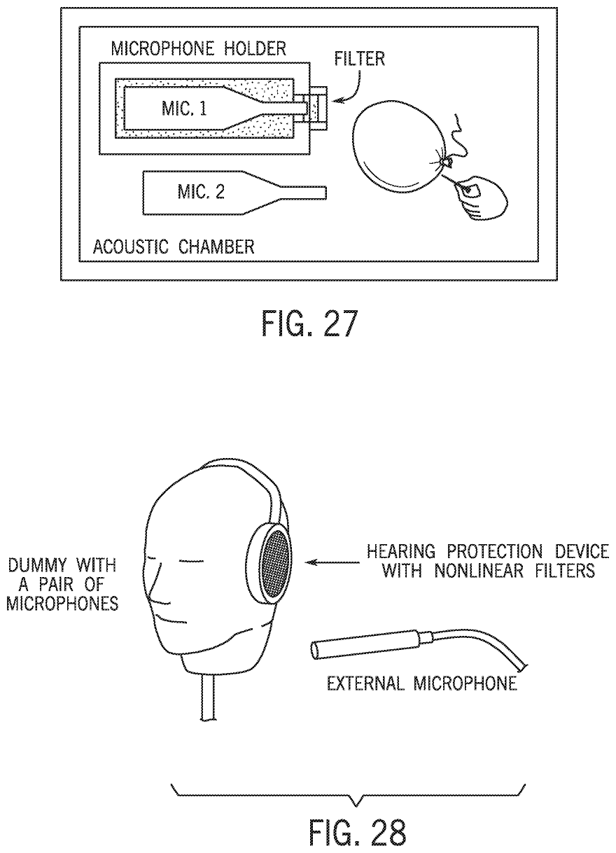

To measure the filters transmission loss for high-SPL input impulses, an experimental test setup was built (FIG. 27). In this test, two microphones with 25 kHz bandwidth are used. Both microphones have higher SPL threshold than the low-SPL test and can measure the impulses with 165 dB peak values. One microphone is placed in a holder which is identical to the one in the low-SPL test. This microphone measures the transmitted sound through the filter (called “Sig”). The microphone recordings are compared in time and frequency domains for the filter loss analysis. The second microphone measures the input sound (called “Ref”). For each filter, the transmission loss is obtained after 10 measurements and averaging to minimize the experimental error. The microphones are either hanged or placed on soft foam to avoid recording of undesired chamber vibrations after the impulse. The microphones tops are only a few centimeter far from each other, so they measure the impulse with about the same intensity and phase. Without the filter mounted, the recordings of the two microphones in frequency domain for a low-SPL input white noise are similar and the difference is mostly less than 5 dB over the entire audible spectrum. However, for a high-SPL impulse input, they record different SPL values (up to 15 dB) at high frequencies (>6 kHz), which means the filter holder has some nonlinear behavior at high frequencies in presence of a blast. The input blasts are generated by popping air-filled water balloons. The SPL peak of the blasts within 10 cm distance from the balloon surface are about 157-163 dB and their rise time is shorter than 0.2 millisecond. The test is performed inside an acoustic chamber with several layers of wood, rubber, and foam to damp the sound. The chamber can provide more than 40 dB isolation when closed.

FIG. 27 shows an experimental setup for testing the acoustic filter for high-SPL input impulses.

### Dummy Test

The filters that are used for making a prototype are already tested for low-SPL and high-SPL transmission loss. The fabricated prototype is also tested using a dummy (FIG. 28). The dummy has a separate microphone on each ear and is designed to perform similarly to human sense of hearing. Both high-SPL test and low-SPL tests are repeated for the dummy to make sure that all the filters in the prototype are functional and the prototype has satisfactory performance. An external microphone records the input SPL for each test. For the low-SPL test, the sound is recorded both with and without the prototype on the dummy and the spectral transmission loss is obtained by subtracting these two recordings in the dB scale in the frequency domain. For the high-SPL test, the microphone records the sound with and without the prototype on the dummy, and in each case the external microphone also records the input SPL. The recordings reveal that how much of the sound attenuation is due to the nonlinear filters.

FIG. 28 shows an experimental setup to test an earmuff prototype that includes an acoustic filter.

### Filter's Performance in Time Domain

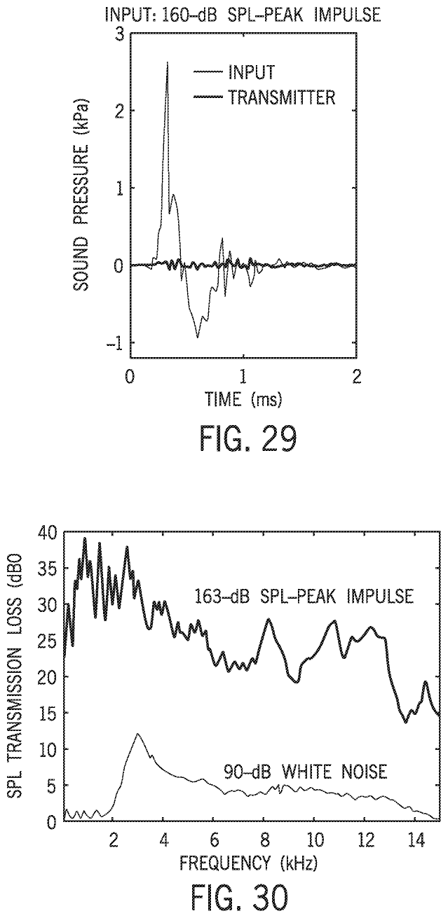

In the high-SPL test the microphones can record the impulse pressure in the time domain. A Fourier Transform of the time-domain recordings reveals their spectral contents as well. FIG. 29 shows a typical recordings of a filter with 10-μm-thick HDPE membrane and 30 μm gap size. The pale blue color (Input) is recorded by Mic. #2 and the dark blue (Transmitted) is by Mic. #1 located inside the holder. The peak of the impulse recorded by Mic. #2 is 163 dB and it is attenuated by more than 30 dB. Mic. #1 also shows some high frequency (14-18 kHz) oscillations. Since this is not within the high-sensitivity audible range of human ears, this may not cause serious issues. But they can be damped by some absorbing foam inside the prototype.

### Filter's Performance Infrequency Domain

Applying a Fourier on the time domain recordings reveal the spectral contents. FIG. 30 shows the spectral transmission loss for the filter that was used for the data in FIG. 29. The different lines represent the loss for the high-SPL impulse and low-SPL white-noise inputs. For the low-SPL test, a low transmission loss in the lower frequencies (0-2 kHz) is more important than the rest of the spectrum. Because this is the spectral range that contributes to the human conversational voice and many other sounds that we hear. The peak at 3 kHz appears in loss of all the filters and should be due to an acoustic resonance which originates from filters inside the holders. The dummy test can confirm this.

### Filter's Typical Nonlinear Transmission

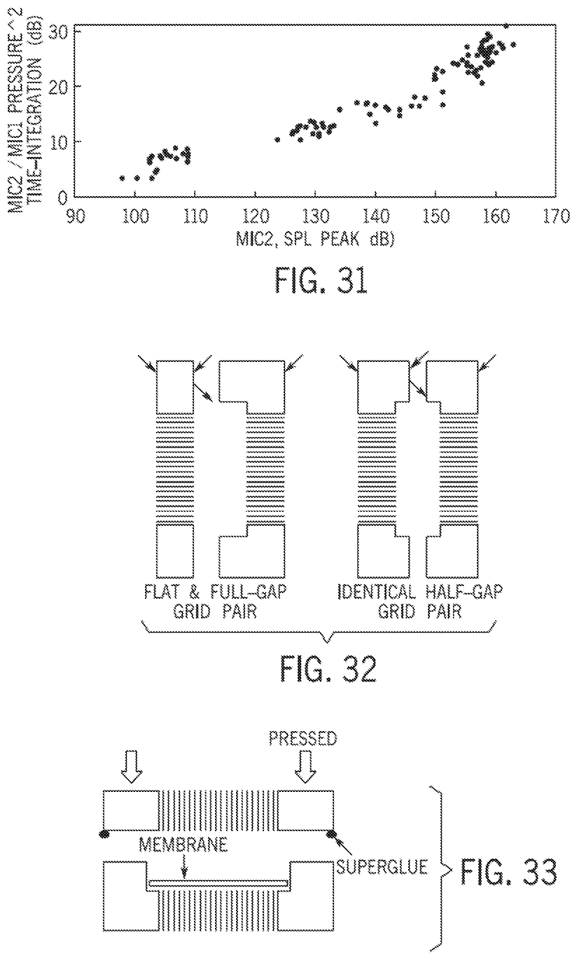

To verify the nonlinear SPL transmission of filters, the SPL of the input sound is gradually reduced, and the transmission is recorded by the microphones. The high SPL input is produced by popping the balloons. To reduce the SPL value, the balloons are filled with less air and popped at a larger distance from the microphones inside the acoustic chamber. The maximum distance is 1 meter and the pops' SPL is measured as low as 100 dB. For each test, the pressure of the sound is measured by two microphones. Comparing the SPL peak values does not show the actual loss of the filter, because based on frequency contents of an input impulse, there might be some random high-frequency spikes in the recordings of Mic1. Such spikes have typically much higher SPL value than the leading side of the impulse and correspond to frequencies of >15 kHz. Since they are very random, and appear only in some recordings, they cannot be used as a reliable parameter to present the filter loss. Instead, we can compare the energy of each impulse measured by the two microphones. The energy of an impulse is proportional to the time integral of an impulse pressure-square. FIG. 31 shows the attenuation of 10s of impulses with different SPL peak values after passing through a filter with similar parameters of the filter used in FIGS. 29 and 30. The attenuation is about 30 dB for a 160-dB SPL-peak input and decreases with reducing the SPL peak value. For a 100-dB SPL-peak impulse, the attenuation is less than 5 dB.

### Fabrication Steps and Assembly of the Filters

1—The grid pairs are fabricated by a 3D printer. The grids can be identical with a half gap on each grid or can be a flat and a full-gap filter. The grids are well polished on a hard polishing surface to avoid surface curvatures. FIG. 32 shows two possible forms of the grid pairs. The surfaces that are marked with the red arrows are the areas that should be finely polished and flat. The inner sides are polished, so that the two pieces match well with no micro-gap between them after assembling. Also, the resolution of the printer may not be sufficient to fabricate a small gap. So, the size of the gap should be checked when polishing. The backsides of the grids are polished because if the gap is being measured with a micrometer caliper, the grids with a not flat back-side may get deformed by the caliper pressure.
2—The membrane should be punched on a hard substrate with the desired size. If the substrate is soft, the membrane edges will get deformed and will cause the membrane to curve. After punching, the membrane can be squeezed between two flat and smooth metallic or any hard plates to remove all possible roughness or small wrinkles. After squeezing, the membrane may stick to one of the plates. It should be lifted off very slowly to avoid any curvature. Two pieces of paper can be placed between the membrane and the plates to make the lift off easier. The smooth and flat membrane should be slowly rubbed over a metallic surface to discharge if there is any electric charge of the membrane during the squeezing.
3—The membrane should be grabbed with a pair of tweezers and placed gently at the middle on a grid. It should be placed inside the gap.
4—some low-viscous superglue should be applied on the inner side edge of the second grid. The amount of glue to be applied should be very small. Otherwise, a large amount of glue may be pushed inside the gap after assembling. Also, a viscous superglue will remain a small gap between the grids. The second grid should be placed on the first grid with the membrane and pressed with hand (FIG. 33). The grids do not need to be assembled with any relative orientation to get the holes aligned.

FIG. 33 shows an acoustic filter prior to coupling the substrates together.

The assembled filter should be tested for the low and high SPL loss. If the filter passes the tests, it can be used in a prototype device.

It will be appreciated by those skilled in the art that while the disclosed subject matter is described above and in the attached Appendix in connection with particular non-limiting examples and examples, the invention is not necessarily so limited, and that numerous other non-limiting examples, examples, uses, modifications and departures from the non-limiting examples, examples and uses are intended to be encompassed by the claims attached hereto.

### Example 2

Earplugs and earmuffs are commonly used to protect against loud noises, but they can interfere with our ability to hear normal sounds such as conversations. To address this issue, a pressure-dependent sound filter has been developed that transmits normal sounds with minimal loss, while instantly and reversibly switching to high attenuation upon the occurrence of a blast or other loud sound impulse. The filter consists of a membrane between grids, where the gap distance between the grids sets a threshold sound pressure level (SPL). The prototype described herein offers 30 dB attenuation for blasts of 160 dB SPL, making it useful for military applications where soldiers need to hear natural sounds while being protected from gunfire and explosions. The technology has a tunable nonlinear threshold, which could improve hearing in other environments with sporadic loud noises.

Noise-induced hearing loss is a widespread problem. According to Occupational Safety and Health Administration (“OSHA”) in U.S. Department of Labor, sounds above 85 dBA sound pressure level (“SPL”) can lead to gradual hearing loss if exposed for more than 8 hours a day. The allowed time of exposure for continuous noises is reduced by half for each 5 dB increase of SPL. Workers are not allowed to be exposed to any continuous noises above 115 dB without hearing protection. Exposure to impulsive or impact noise should not exceed 140 dB peak pressure, a typical pain threshold. A typical gunfire can impose 155-160 dB impulse noise to the shooter. Even a single blast, when transmitted to the inner ear, can cause instant and irreversible damage to hair cells. Warfighters and other supporting personnel (e.g., medics) are constantly exposed to the high risk of blast-induced hearing loss, making hearing loss one of the most prevalent service connected disabilities. As of 2020, more than 1.3 million veterans in the United States received disability compensation for hearing loss.

Using hearing protection devices can effectively reduce the risk of noise-induced hearing loss, although currently available devices have certain drawbacks. Passive earplugs, such as foam earplugs and “combat” earplugs with sound-blocking caps, can help reduce hearing loss. However, fixed attenuation earplugs can decrease the wearer's ability to hear normal sounds, such as conversations, and also make it difficult to determine the direction of sounds, which is crucial for situational awareness during military operations. These limitations often lead soldiers to avoid using hearing protection devices altogether. Additionally, when the manual caps are open, wearers can hear natural sounds, but are left exposed to unpredictable blasts without protection.

To address the limitations of fixed attenuation or manually adjusted earplugs, researchers have been exploring the use of nonlinear transmission filters. These filters include orifice filters and sound-induced movable plugs, which have shown SPL-dependent transmission but with limited dynamic range and relatively high background attenuation. Orifice earplugs have been used by the U.S. military, but nearly 300,000 military veterans have filed lawsuits alleging that the dual-ended Combat Arms earplugs were defective and failed to protect them from hearing damage. Active electronic hearing protection devices also exist, which provide electronics-controlled variable transmission. However, these devices require power sources, are more expensive, and do not reproduce all the qualities of natural sound waves. The inability to hear natural sounds negatively impacts sound recognition and localization. Efforts are ongoing to develop more effective and practical hearing protection devices to reduce the risk of noise-induced hearing loss.

Here a novel acoustic filter is presented that is capable of transmitting normal sounds with minimal loss while attenuating high-intensity noises. This nonlinear transmission is achieved through the use of a structure-induced membrane restriction. This technology, called the Sound High-Energy Limiting Device (“SHIELD”) below, has a well-defined threshold for input SPL above which transmission is restricted. The maximum transmitted energy is limited to below 130 dB SPL against 160 dB incoming blasts. Prototypes have been developed for both earplugs and earmuffs that can switch instantly to high attenuation mode in the event of blasts above the threshold, blocking them from entering the ear. The devices then instantly restore to the normal high transmission mode, allowing people to hear natural sounds while protecting their ears from high SPL noises.

### 2.1. Principle of Restriction-Induced Nonlinear Attenuation

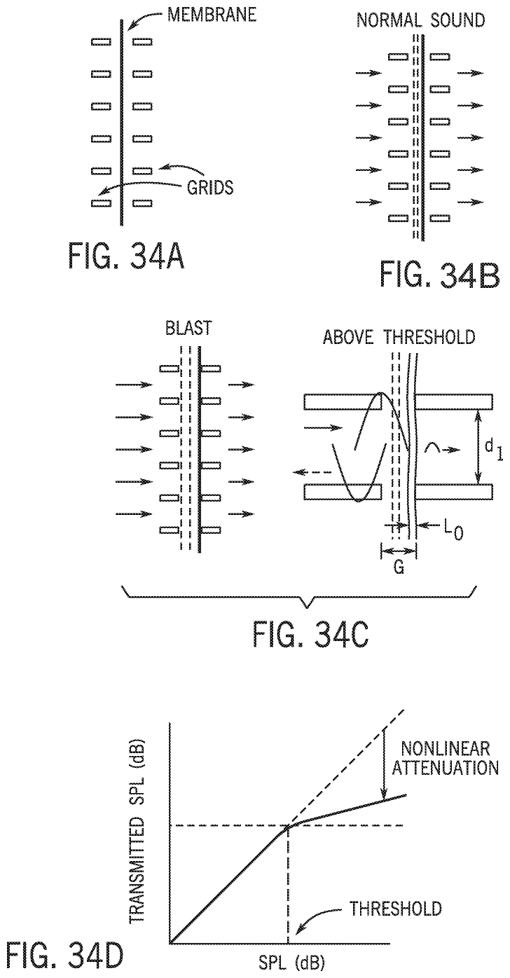

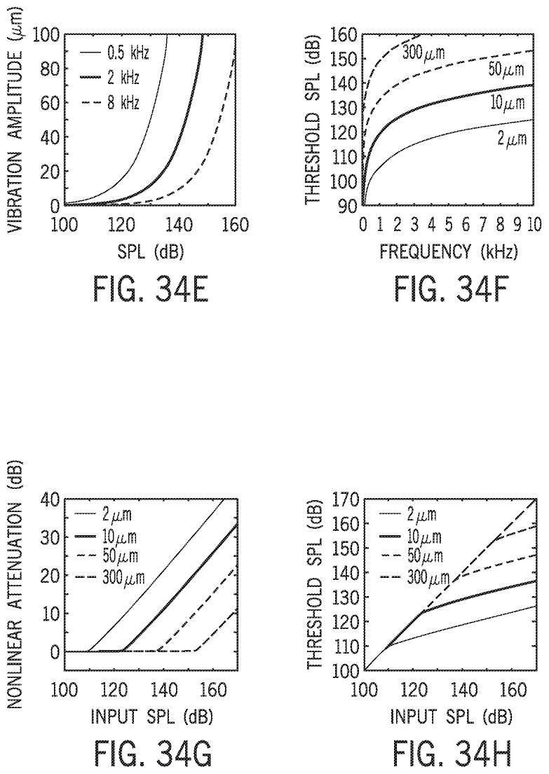

The design concept is illustrated in FIG. 34A. It includes a thin membrane placed between a pair of grids. For normal sound, the membrane vibrates freely without obstruction, resulting in low attenuation of sound transmission (FIG. 34B). When a blast input causes air displacement greater than the gap distance between the grids, the vibration of the membrane is restricted, thereby reducing sound transmission (FIG. 34C). Two main assumptions are made: (1) that the membrane and grids allow for minimal attenuation of normal sound transmission, and (2) that the membrane-grid structure produces considerable attenuation when the membrane makes firm contact with one of the grids. These two conditions are described in the next section. Under these assumptions, the acoustic transmission through the structure exhibits a nonlinear curve with a sharp threshold, as illustrated in FIG. 34D. The threshold occurs at an SPL that is high enough to cause the membrane to make full contact with the grid. Above the threshold, the transmitted SPL exhibits a greatly reduced slope, as we will demonstrate below.

FIG. 34A-34H show the Principle of nonlinear SHIELD attenuation and theorical prediction. FIGS. 34A-D show the design concept. FIG. 34A shows a schematic of the SHIELD filter. FIG. 34B shows the membrane's free vibration for a low-SPL input. Arrows indicate sound waves. FIG. 34C shows the membrane's obstructed vibration for a high-SPL impulse above the threshold. G is the grid separation, L₀ is the thickness of the membrane, and d₁ is the diameter of holes in the grid. Parabolic profiles illustrate incoming, transmitted, and reflected impulses. FIG. 34D shows a schematic of the expected nonlinear transmission curve with a sharp transition at the threshold and increasing attenuation above the threshold. The dashed line shows the transmission of an open membrane without restriction. FIGS. 34E-H show theoretical predictions. FIG. 34E shows a graph of the vibration amplitude of the air by SPL for three different frequencies. FIG. 34F shows a graph of the threshold SPL for monotonic waves of different frequencies for various gap sizes G−L₀. FIG. 34G shows a graph of the restriction-induced loss as a function of peak SPL at 2 kHz for different gap sizes. FIG. 34H shows a graph of the input-output curves from the nonlinear attenuation in FIG. 34G.

Consider a sound wave with peak pressure P and frequency f. The amplitude of vibration of air particles can be calculated using the equation (1):

$$ξ = \frac{P}{{Z}_{air} ω} \qquad ( 1 )$$

In equation (1) Zₐᵢᵣ is acoustic impedance of air (˜415 Pa·s/m³) and a is angular frequency. FIG. 34E illustrates the vibration amplitude as a function of peak SPL, defined as

$$20 {log}_{10} \frac{P}{{P}_{r}}$$

in decibels (dB), where Pᵣ=20 μPa is the reference pressure by definition. For a free, unrestricted membrane, the transmission is ξT^{0.5}, where T represents energy transmission and can be 1 when the membrane is thin and lightweight enough. Restriction-induced attenuation occurs when ξ exceeds the distance between the membrane and grid, which can vary between 0 and G depending on the initial location and flatness of the membrane. The threshold pressure is defined as shown in equation (2):

$${P}_{th} = {Z}_{air} ω g / \sqrt{2} \qquad ( 2 )$$

In equation (2) g=G−L₀ is the nominal gap size. FIG. 34F shows the threshold SPL (SPLₜₕ) as a function of frequency for different gap sizes. The threshold SPL decreases at lower frequencies. For example, with g=10 μm, SPLₜₕ=120 dB for 1 kHz, SPLₜₕ=100 dB for 100 Hz, and SPLₜₕ=80 dB for 10 Hz. Typical blasts have peak frequencies in the 1-10 kHz range.

Consider a blast wave that is incident at time t=0, with sound pressure described by p=P sin(ωt). As the pressure rises, the membrane is pushed forward until it hits the grid at t=t₀. The time of contact to is calculated as follows:

$$cos ( ω {t}_{c} ) = 1 - \frac{{P}_{th}}{\sqrt{2} P} for P \geq \frac{{P}_{th}}{\sqrt{2}} .$$

After t₀, there is negligible transmission of the blast through the restricted membrane-grid structure. The transmitted energy during the pressure uprising period is given by

$${E}_{out} = {\int }_{0}^{{t}_{0}} {p}^{2} dt / {Z}_{0} .$$

During this period, the total input energy is

$${E}_{in} = {\int }_{0}^{T / 2} {p}^{2} dt / {Z}_{0} .$$

The restriction-induced attenuation is given by

$$Att . ( dB ) = - 10 {log}_{10} \frac{{E}_{out}}{{E}_{in}} This outputs \frac{{E}_{ou}}{{E}_{in}} = \frac{{cos}^{- 1} ( 1 - \sqrt{\frac{2}{x}} )}{π} - \frac{{( 2 \sqrt{2} x - 2 )}^{0.5} ( x - \sqrt{2} )}{π {x}^{2}} ,$$

where x=P/Pₜₕ is the normalized pressure, and

$$\frac{{E}_{out}}{{E}_{in}} = 1 when P < {P}_{th} / \sqrt{2} .$$

This function is plotted in FIG. 34G. As a simple approximation, the attenuation can be written as equation (3):

$$Att . ( dB ) \approx 0.75 ( {SPL}_{in} - {SPL}_{th} ) \qquad ( 3 )$$

In equation (3),

$${SPL}_{in} = 20 {log}_{10} \frac{P}{{P}_{r}} and {SPL}_{th} = 20 {log}_{10} \frac{{P}_{th}}{{P}_{r}} .$$

Finally, the output SPL is then given by equation (4):

$${SPL}_{out} \approx {SPL}_{in} - 0.75 ( {SPL}_{in} - {SPL}_{th} ) \qquad ( 4 )$$

The input-output response curve is plotted in FIG. 34H for different threshold conditions. Above the threshold, the curve has a linear slope of 0.25 except for very near the threshold.

### 2.2. Theoretical Optimization of Design Parameters

The above analysis used two premises: First, both a freely vibrating membrane and grid can have high transmission. Second, the membrane in contact with the grid can induce high loss. Here we evaluate these conditions.

### (i) Membranes at Low SPL

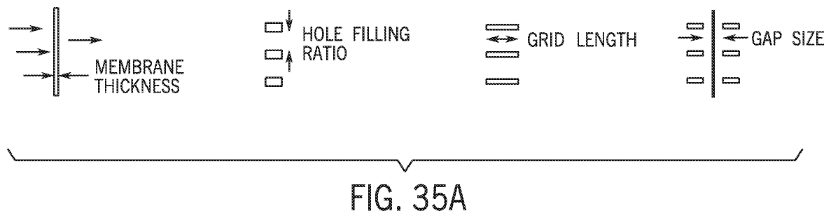

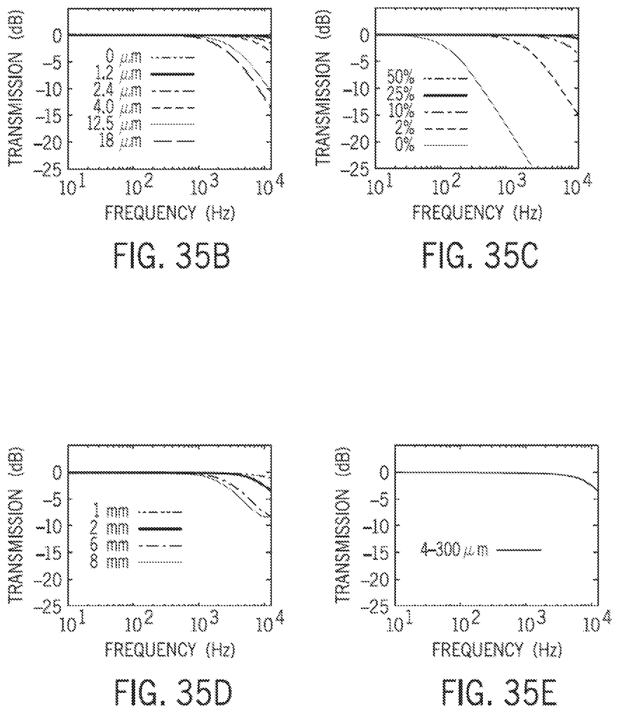

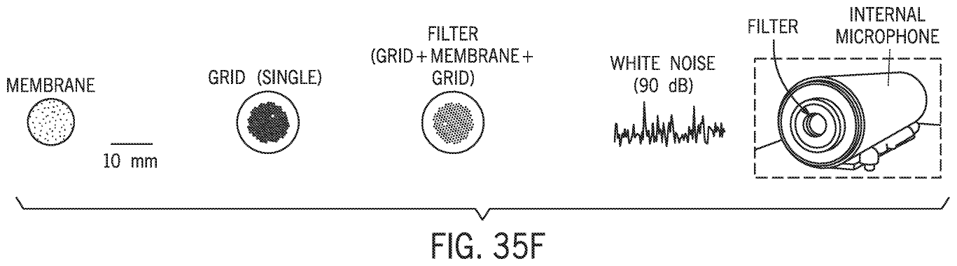

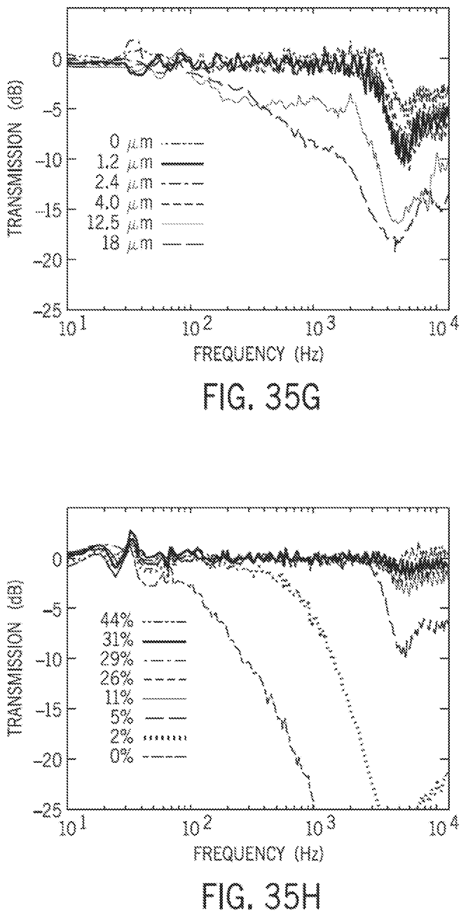

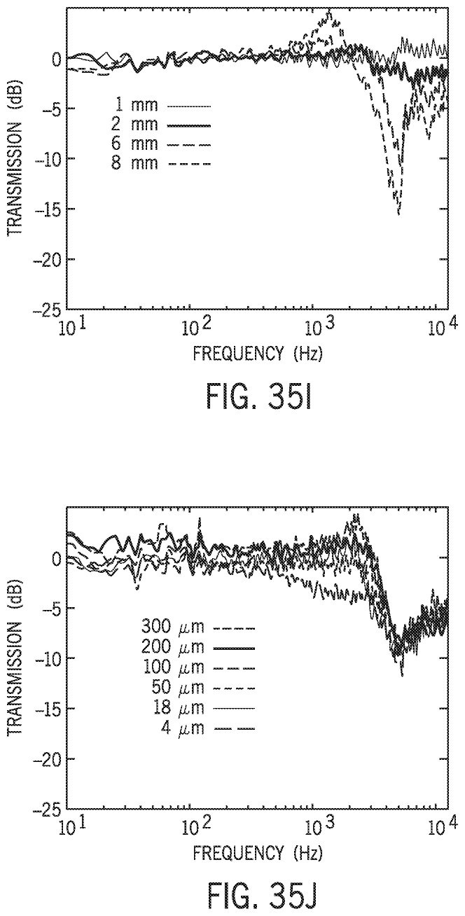

Consider a sound wave incident on a freestanding, flat membrane with a uniform thickness L₀ (FIG. 35A). From the multi-layer interference theory, the transmission is

$$T = {( 1 + \frac{4 R {sin}^{2} ϕ}{{( 1 - R )}^{2}} )}^{- 1} , where R = \frac{{( {Z}_{m} - {Z}_{air} )}^{2}}{{( {Z}_{m} - {Z}_{air} )}^{2}}$$

is the reflectivity at the air-membrane interface. Here, Zₘ=ρₘvₘ is the acoustic impedance of the membrane material with density ρₘ and acoustic velocity vₘ·φ=ωL₀/vₘ is the phase delay across the membrane. FIG. 35B plots the transmission in decibels, 10 log₁₀ T, for aluminum membranes (vₘ=6300 m/s and ρₘ=2700 kg/m³) with thicknesses ranging from 1.2 to 18 μm, 0 μm corresponds to a pair of grids without a membrane. Since φ<<1 and Zₘ>>Zₐᵢᵣ, the formula is reduced to

$$T ( dB ) = - 10 {log}_{10} ( 1 + \frac{{ω}^{2} {m}^{2}}{{( 2 {Z}_{a i r} )}^{2}} ) ,$$

where m=ρₘL₀ is the mass density per unit area. Acoustic loss less than 3 dB is met below 10 kHz if L₀<4.9 μm for aluminum and L₀<13 μm for less dense materials.

FIG. 35A-J shows the transmission of the SHIELD filters with respect to low SPL sounds. FIG. 35A shows four different design parameters. FIG. 35B shows theoretical plots of transmission for different membrane thicknesses. FIG. 35C shows theoretical plots of transmission for different hole filling ratios for a grid size of 50 μm. FIG. 35D shows theoretical plots of transmission for different grid lengths for a hole filling ratio of 0.2. FIG. 35E shows theoretical plots for different gap size for a grid size of 50 μm and filling ratio of 0.2. FIG. 35F shows illustrations of an aluminum membrane, a grid, an assembled filter, and the transmission measurement setup for white noise at 90 dB SPL. FIG. 35G shows the experimental results for different thicknesses of membrane placed between grids (Lₕ=1 mm, S=20%, G=50 μm, D₀=15 mm). FIG. 35H shows the experimental results for different hole filling ratios. FIG. 35I shows the experimental results for different grid lengths. FIG. 35J shows the experimental results for different grid separations G. Notably, for a solid disk (S=0%) shown in FIG. 35H, the attenuation was 60 dB at 4.3 kHz but <5 dB below 100 Hz. This highlights the general difficulty of blocking low frequency sounds.

### (ii) Grids at Low SPL

The acoustic impedance of a hollow pipe is inversely proportional to its cross-sectional area. Consider a single, disk-shaped grid with an outer diameter D₀ having N holes with a smaller diameter of d₁. To estimate transmission, the grid may be regarded as a pipe of the same area, with a diameter D₁=\sqrt{N}d₁. The reflectivity at the proximal end when the diameter changes from D₀ to D₁ is equal to

$${R}_{h} = \frac{{( 1 - S )}^{2}}{{( 1 + S )}^{2}} , where S = {( {D}_{1} / {D}_{0} )}^{2}$$

is the hole filling ratio. The same reflectivity occurs at the distal end of the hole. A grid with a length Lₕ forms Fabry-Perot interference, and its transmission is given by

$$T = {( 1 + \frac{4 {R}_{h} {sin}^{2} {ϕ}_{h}}{{( 1 - {R}_{h} )}^{2}} )}^{- 1} , where ϕ = ω {L}_{h} / {v}_{air} and {v}_{air} = 346 m / s .$$

This transmission through a single grid is plotted as a function of S in FIG. 35C, and as a function of the grid length Lₕ in FIG. 35D. In FIG. 35E, transmission through assemble filters is plotted, where the attenuation of the three elements—the membrane (L₀=2.4 μm), entrance grid, and exit grid (S=0.2)—have been simply added.

For small gaps with g from 4 to 300 μm, the sound wave propagation is not affected by the gap as long as the membrane is free to vibrate. This result supports the validity of the first premise stated earlier.

(iii) Transmission in the Motion-Restricted State.

The dynamics of the membrane inside the gap, once it makes physical contact with a grid, may be complex due to its coupling with aerodynamics. However, it can be modeled as a circular plate with bending stiffness

$$D = \frac{{EL}_{0}^{3}}{12 ( 1 - {v}^{2} )} ,$$

where E is Young's modulus and ν is the Poisson's ratio of the membrane. The structure has a fundamental resonance at

$${ω}_{0} = α \sqrt{\frac{D}{{ρ}_{w} {L}_{0}}} {( \frac{2}{{d}_{1}} )}^{2} ,$$

where α is 5 to 10 depending on the boundary condition. The transmission is given by

$$T = {\vert 1 - i \frac{{ρ}_{w} {ω}_{0}^{2} {L}_{0} ( 1 - {ω}^{2} / {ω}_{0}^{2} )}{2 {Z}_{air} ω} \vert}^{- 2} .$$

To ensure high loss, the resonance frequency should be outside the signal range. In this case,

$$T {\to }^{ω ≪ {ω}_{0}} \sim {( \frac{{d}_{1}}{2} )}^{8} {( \frac{{z}_{air} ω}{{EL}_{0}^{3}} )}^{2} .$$

The critical parameter is d₁, which must be sufficiently small. For 2 kHz, the transmission loss (10 log₁₀ T) is −36 dB at d₁=0.5 mm, −24 dB at d₁=0.8 mm, and −12 dB at d₁=1 mm. Considering that the sound conduction through the bone is typically around −30 dB¹⁴, a high attenuation criterion of −30 dB can be set. The second premise is satisfied when d₁<˜0.6 mm.

### 2.3. Experimental Verification Against Low SPL Sound

Based on the design guidelines above, various membranes and grids were produced and tested with different dimensions and materials. Initially, thin, tough, elastic polymeric membranes were examined like thermally pressed high-density polyethylene or cutouts from plastic bags. They work well, as long as their thickness was less than 5-10 μm. However, the ultra-flexible membranes were difficult to handle and often came into contact with grids due to bending, resulting in around 5 dB background attenuation. It was found that one of the better options was aluminum foils of different thicknesses. For grids, we utilized thin metal tubes or capillaries stacked in a large tube, but one of the better options was to 3D print hard resin with a hexagonal array of holes, as shown in FIG. 35F. To control the separation distance between the grid, a thin spacer material attached to one of the grid surfaces was used. After inserting a circular aluminum foil cut to size, glue was applied outside the grids to seal the gap space. This assembly creates a SHIELD filter. To measure transmission, the filter was mounted on the ring opening of a metal housing that contains an internal microphone as shown in FIG. 35F. Another microphone was positioned externally right next to the filter to record input signals.

FIG. 35G depicts the measured transmission of white noise sound at 90 dB SPL through six devices with different aluminum membrane thicknesses but identical grid dimensions (G=50 μm, d₁=0.5 mm, S=20%. Lₕ=1 mm, D₀=15 mm). The overall trend agrees with the prediction in FIG. 35B. The transmission dips around 6 kHz and the increases above 6 kHz are partly caused by resonance in a Helmholtz cavity between the filter and microphone. When the cavity volume was altered, the resonance shifted but could not be moved further above 6 kHz due to mechanical constraints. FIG. 35H-J illustrate the measurements when the hole size, length, and separation of the grids were modified for the same membrane thickness of 2.4 μm. The overall trends align with the theory in FIG. 35C-E.

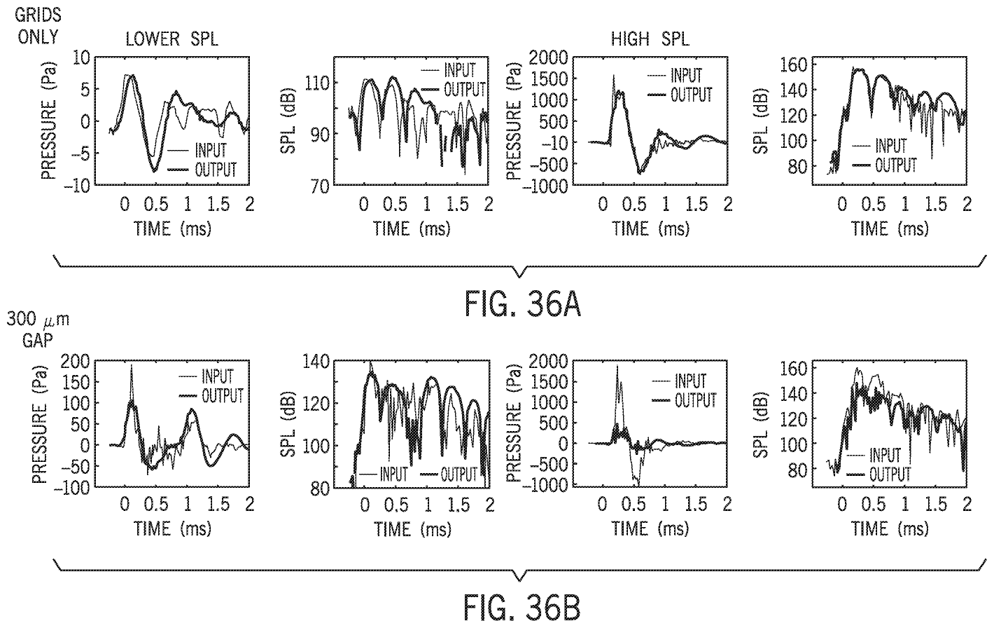

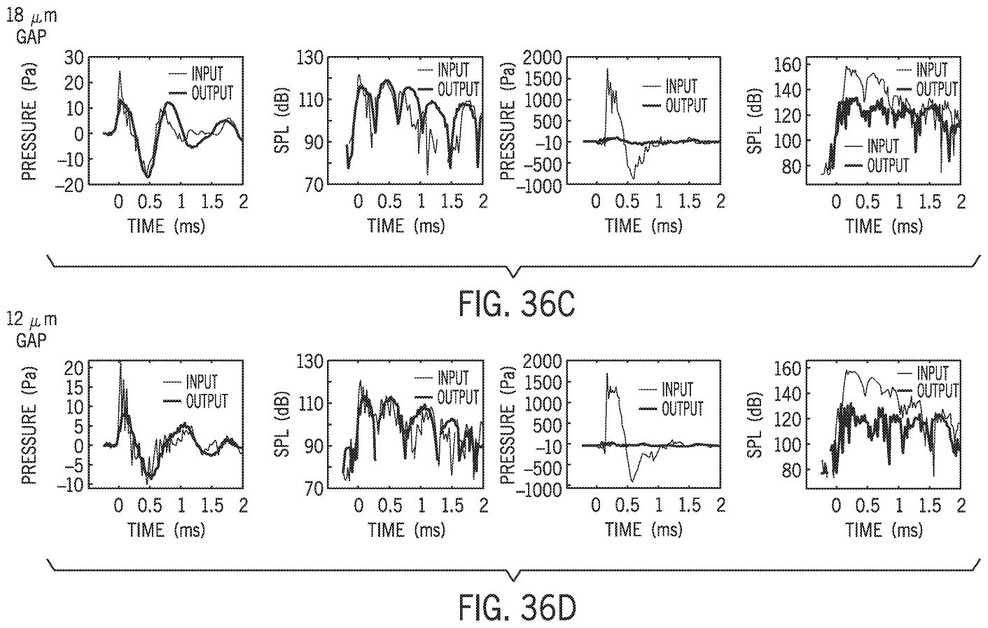

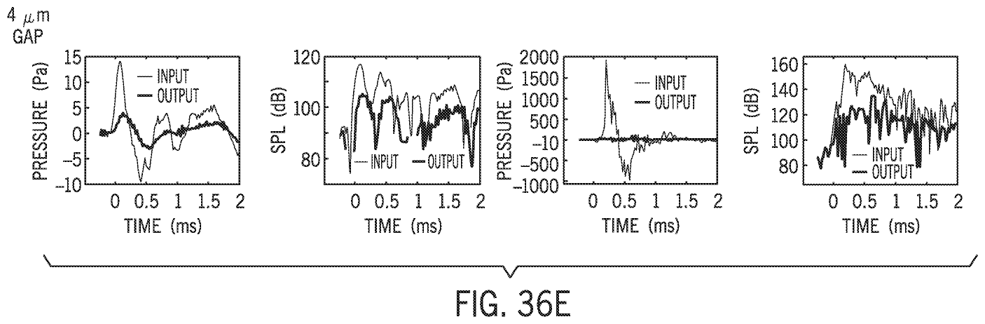

To characterize nonlinear attenuation, blasts were generated using balloon pops. FIGS. 36A-E show representative time-domain recordings from five filters made with different grid separations. Lower SPLs were obtained with balloon 7 pops at distance, and the highest SPLᵢₙ up to 160 dB was produced by balloon pops right next to the devices. In all devices, the transmission was high for low-SPL impulses. For high-SPL, significant attenuation of about 30 dB was observed in devices with small gap sizes below 20 μm (FIGS. 36C-E). A 300-μm gap yields modest nonlinear attenuation (FIG. 36B). A pair of grids without a membrane shows high transmission even at 160 dB input (FIG. 36A). These results agree well with the theory presented earlier.

FIGS. 36A-E show impulse responses measured experimentally from 5 samples. The light curves represent input blasts recorded by the external microphone, while the darker curves represent transmitted signals recorded by the internal microphone. The first and second graphs for each FIG. 36 (e.g., FIG. 36A) show responses to lower peak SPLs (below threshold) in linear and dB scales, respectively. The third and fourth graphs for each FIG. 36 (e.g., FIG. 36A) display data for 160 dB peak SPL. Nonlinear attenuation is absent in FIG. 36A, is modest in FIG. 36B, but becomes increasingly evident as the gap size is reduced.

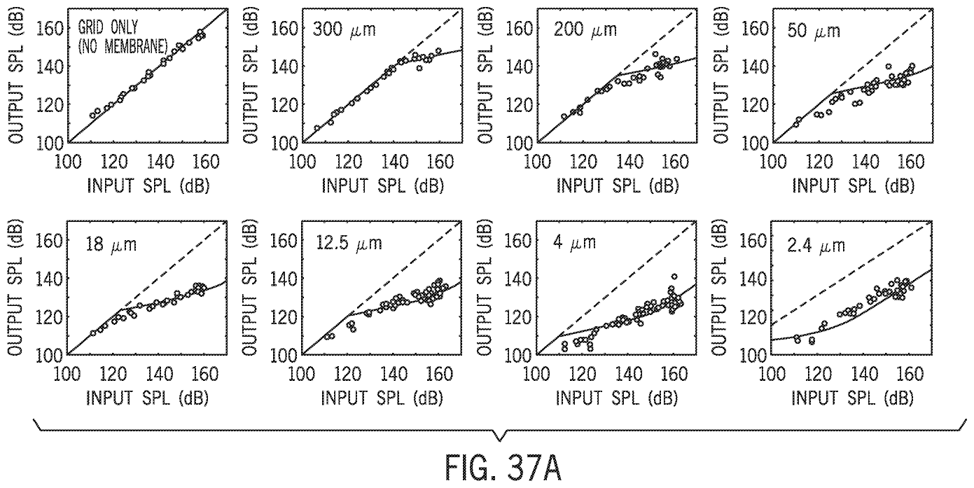

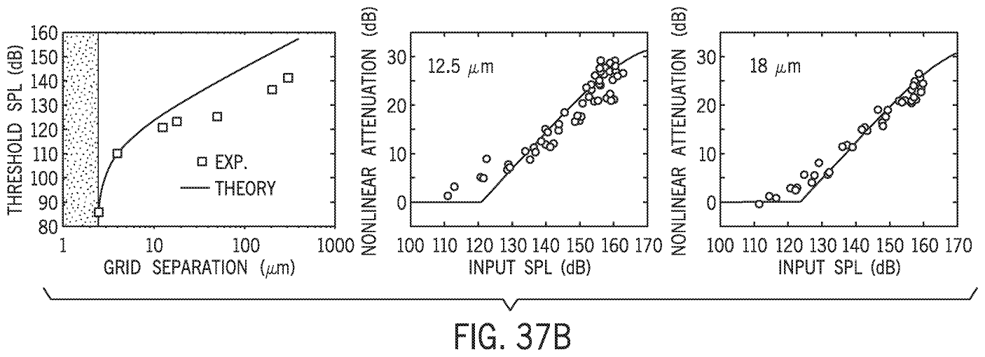

The time-domain traces were recorded of numerous different balloon pops generated at varying distances and analyzed the total loss,

$$Loss = 10 {log}_{10} {\int }_{0}^{Δ t} {p}_{out}^{2} dt - 10 {log}_{10} {\int }_{0}^{Δ t} {p}_{in}^{2} dt ,$$

where the integration time was chosen to either Δt=0.34 μs, which approximately corresponds to the duration of the first positive pressure peak of the input blast, or 0.68 μs, which spans the first positive and negative pressure oscillations. Both choices produced quite similar results. Then, the transmitted SPL was calculated by SPLₒᵤₜ=SPLᵢₙ(peak)−Loss. The reason for using this method is that while the peak SPL of the incoming blast is well-defined, the attenuated output does not usually have a defined peak coincident with the input peak, as evident in FIGS. 36C-E. FIG. 37A shows the analysis result for a total eight samples.

First of all, the empty pair of grids without a membrane show nearly lossless, linear transmission over the entire SPL range. The Reynolds number of air flow through the holes is given by Re=ρₐᵢᵣvd₁/μ, where ρₐᵢᵣ=1.29 km/m³, v=p/Zₐᵢᵣ and μₐᵢᵣ=18.5 μPa·s are the density, velocity, and viscosity of the air, respectively. With d₁=0.5 mm, Re=10 for at 135 dB SPL and Re=180 at 160 dB SPL. At the exit of the distal grid, all pressure waves through the array of holes are combined. This interference minimizes the turbulence at the exit, avoiding the open cavity or orifice effect. The experimental data supports that there are no nonlinearity-induced losses through the grid holes, at least up to 160 dB peak SPL.

As the grid separation decreases, it becomes clear that the threshold SPL also decreases. To fit the data, we used Eq. (4) and added a constant background transmission of −33 dB to account for any leakage, such as non-airborne transmission through the plastic walls of the grids. This modification was necessary to explain the experimental data, which showed saturation of nonlinear attenuation beyond 30 dB dynamic range in 4 μm and zero gap devices. In the fitting, G was the only variable, and we obtained the best fits with G=2.5, 4, 8, 10, 12, 37, and 60 μm for the experimental design values of 2.4, 4, 12.5, 18, 50, 200, and 300 μm, respectively (g=G−2.4 μm) between experiments and theory. FIG. 37B, in the first graph, shows SPLₜₕ as a function of G, indicating reasonable correspondence between experiments and theory. However, the apparent discrepancy in larger grid separation devices is not fully understood but speculated to be related to the position of the membrane and air flow around it. Finally, in the second and third graphs of FIG. 37B, the Loss data was plotted in two representative devices that have optimal gap sizes for our intended goal.

FIGS. 37A and B show experimental results of impulse responses. FIG. 37A shows input-output curves of 8 different devices with different gap sizes. Experimental data shown as circles, with solid curves indicating the best fit with Eq. (4) after adding a background transmission of −33 dB. The background transmission (leakage) accounts for the saturation of nonlinear attenuation when SPLᵢₙ−SPLₜₕ>40 dB. The first graph of FIG. 37B shows a measured threshold SPL values for different gap sizes. Experimental data shown as squares, with a theorical fit based on Eq. (2) shown as a curve. The second and third graphs of FIG. 37B show attenuation measured as a function of input SPL for two representative devices in FIG. 37A. Experimental data shown as circles, with solid curves indicating the best fit with Eq. (3) after adding the background transmission.

### 2.5. SHIELD Earplug

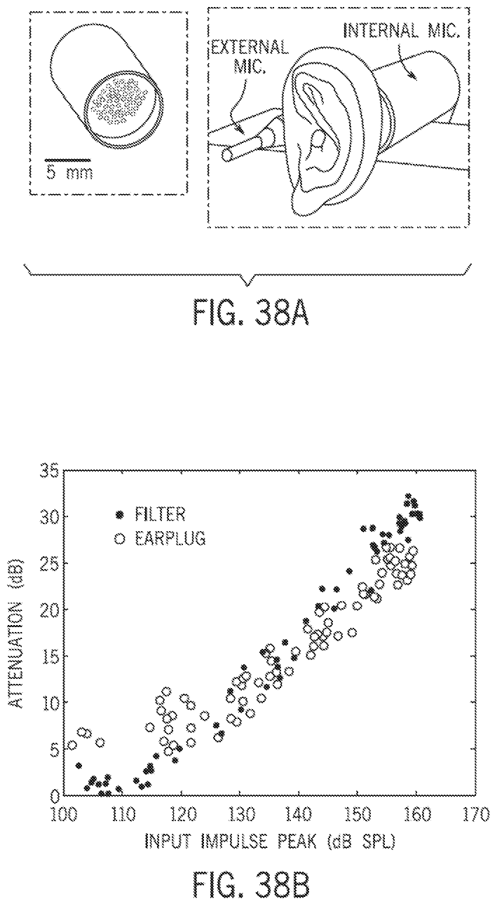

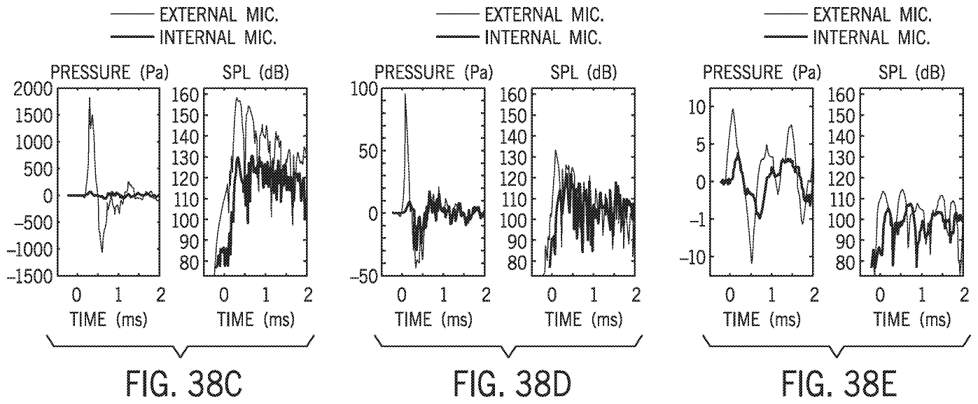

Filter were fabricated with a smaller outer diameter D₀=8 mm and G=12 μm (all other parameters remained the same as before). FIG. 38A shows a prototype earplug with one of the filters inside a metal housing. The transmission was measured with respect to balloon pop blasts using a setup consisting of inserting the earplug into a model ear and exposing to the balloon pop blasts, while recording the transmitted signals using an external microphone. FIG. 38B presents the result, in comparison with the performance of the filter measured outside the earplug assembly. It exhibits nonlinear attenuation of 23-27 dB at 155-158 dB input SPL. Representative time-domain traces at 159-, 134-, and 114-dB inputs, showing high, moderate, and low attenuations, respectively, are displayed in FIG. 38C-E.

FIG. 38A-E show SHIELD earplugs and results of tests. FIG. 38A shows an illustration of a prototype earplug and the measurement setup using a model ear. FIG. 38B shows a graph of a comparison of experimentally measured attenuation of the earplug (open circles) within the ear model and the performance of the bare filter itself (solid circles). FIGS. 38C-E show time-domain recordings of the incoming and transmitted pressure levels for FIG. 38C high-, FIG. 38D moderate-, and FIG. 38E low-SPL blasts.

### 2.6. SHIELD Earmuff

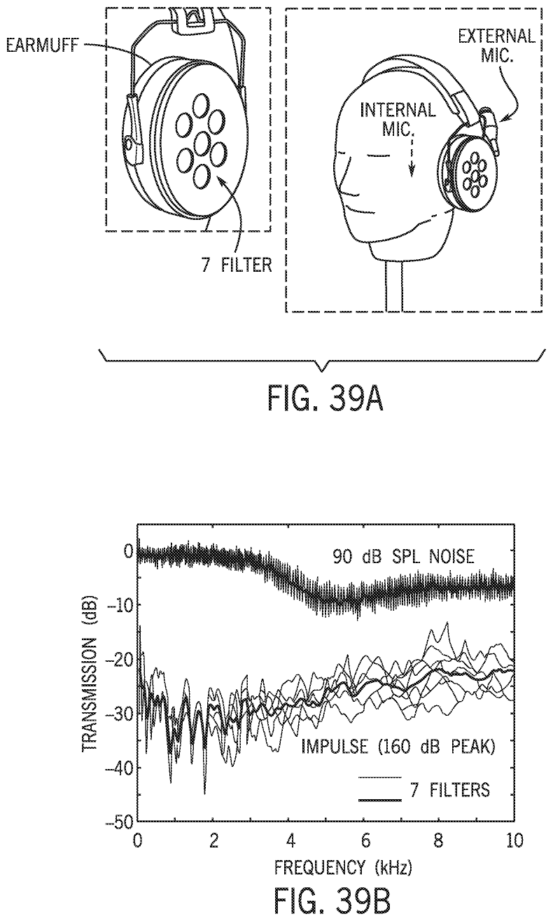

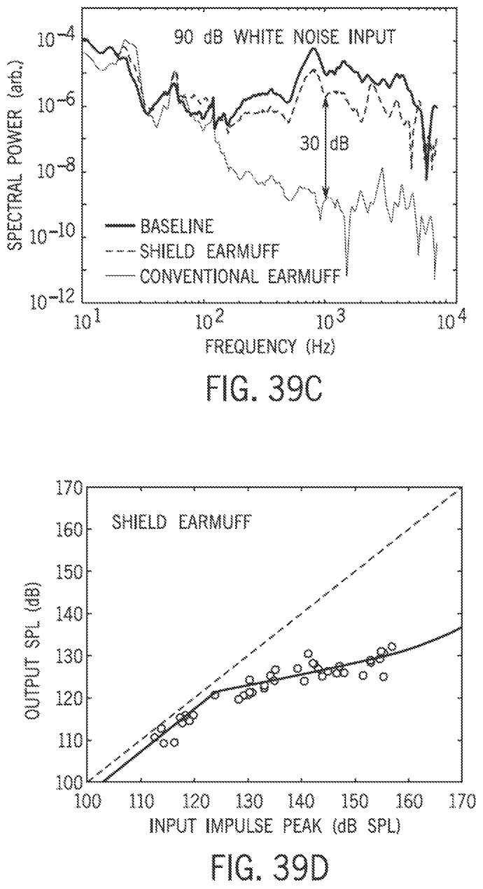

A prototype earmuff was fabricated by taking a commercial earmuff, removing the original plastic caps and all internal materials, and installing an 8-mm thick acrylic plate containing 7 filters (D₀=15 mm, G=12 μm), as shown in FIG. 39A. Each filter was inserted into a conduit hole prepared in the plate. To measure the transmission of the earmuff, a dummy head microphone (Neumann KU 100) was used. All 7 filters showed low-loss transmission for low SPL noise and high nonlinear attenuation of 30 dB for 160 dB blasts (FIG. 39B). A fully assembled prototype earmuff exhibited some insertion loss between 300 Hz and 10 kHz, whereas an unmodified earmuff had much greater fixed attenuation in the same frequency range (FIG. 39C). The loss at low frequencies below 100 Hz was nearly negligible in both devices. As before, the transmission against balloon pop blasts was measured while the earmuff was mounted on the dummy. FIG. 39D plots the data, along with a theoretical fit, demonstrating the earmuffs output limiting function.

FIG. 39A-D shows SHIELD earmuffs and results from tests using the earmuffs. FIG. 39A shows an illustration of the prototype. FIG. 39B shows transmission profiles of the 7 individual filters tested separately against 90 dB white noise (i.e., upper) and 160 dB blasts (i.e., lower). Thicker curves represent the mean values of the 7 filters. FIG. 39C shows the power spectra of the internal microphone compared between open ear (i.e., baseline), the SHIELD earmuff, and an unmodified commercial earmuff against 90 dB white noise. FIG. 39D shows the transmission characteristic of the prototype earmuff. Experimental data are represented by circles, and the theoretical fit is shown by the solid curve, indicating nonlinear transmission and a small insertion loss of 3 dB.

### 2.7. Effects of Wind

Wind at a velocity v_{wind} generates wind pressure,

$${P}_{wind} = \frac{1}{2} {ρ}_{air} {v}_{wind}^{2} .$$

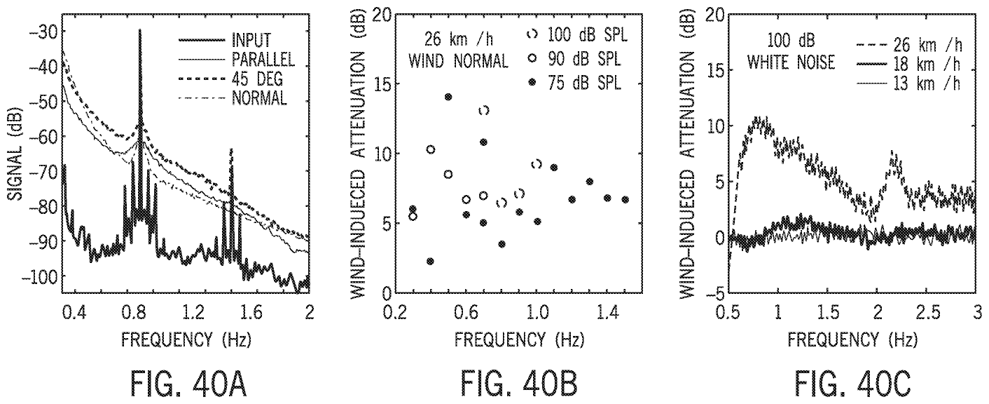

If this pressure is exerted entirely on the membrane, it could push it onto the grid and impede sound transmission. The earmuff prototype was tested against wind generated from an air nozzle. FIG. 40A shows the result for a 700 Hz monotone input for wind at 26 km/h in three different directions. Wind noise is dominant at low frequencies, decreasing at a rate of 26-30 dB per octave. Similar noise is experienced by the average cyclist. Besides this typical wind noise, increased attenuation was measured when the wind is normal to the device, which varied in a range from 2 to 14 dB over frequency, modestly increasing with the input SPL (FIG. 40B). Wind blowing parallel or at 45 deg to the device did not affect transmission. Lower wind speeds than 18 km/h or 16 Pa in pressure did not affect transmission (FIG. 40C). For blast impulses, the air pressure is applied entirely to the membrane within a short duration. However, the wind pressure is much more slowly developed and may be counteracted by pressure built up within the device and in the closed space behind the device, such as the ear canal. Interestingly the threshold wind pressure (118 dB) is similar to the device's nonlinear threshold around 124 dB against blasts, although a concrete model is not available for the behavior against winds.

FIG. 40A-40C shows the effect of wind on SHIELD earmuff transmission. FIG. 40A shows microphone data for a 700 Hz monotone input at 100 dB SPL (i.e., input). Wind at a speed of 26 km/h induces negligible for a wind direction parallel to the device (i.e., parallel), 1.5 dB for the wind blowing at 45-degree angle to the device (i.e., 45 deg), and 14 dB when the wind hits the device at a normal angle (i.e., normal). FIG. 40B shows wind-induced attenuations measured for various input SPL and frequencies for wind blowing at a normal angle to the device at 26 km/h. FIG. 40C shows attenuation at different wind speeds measured using white noise with 100 dB SPL.

This research has unveiled a new passive mechanism for controlling sound transmission that provides protection against loud noises while still allowing for the perception of normal, natural sounds. The SHIELD prototypes, have a low insertion loss of <3 dB over the auditory frequency range, which is 5-10 dB lower than commercial orifice filters, and a nonlinear attenuation of 30 dB, which is 10 dB higher than previous orifice filters. Attenuation greater than 30 dB is typically not needed as the bone conduction efficiency is around −30 dB. Against peak blasts of 160 dB, the SHIELD devices limit the transmission to 130-135 dB, which is sufficient to protect the ear of a soldier during shooting below the pain threshold of 140 dB. The SHIELD devices have an instant response faster than our microphone bandwidth of 23 μs against high-pressure blast peaks, and the normal low-loss mode is instantaneously resumed after the blasts.

A helmet was modified with SHIELD filters and preliminary human tests were conducted on both earmuff and helmet prototypes. The tests revealed that hearing environmental sounds through the filters is similar to the natural experience of naked ears. However, hearing one's own voice while wearing the prototypes is comparable to speaking with loosely hand-covered ears due to suboptimal sound collection. This preliminary study suggested that adding pinna-mimicking reflectors or tilting the filter orientation toward the front can enhance sound collection for the wearer's voice. Human tests against loud blasts are yet to be conducted.

While the nonlinear threshold at 120-130 dB is appropriate for anti-blast protection, it should be possible to optimize filters for lower threshold SPL than 110 dB by improved fabrication precision of the grids. Such low threshold devices may have broader applications for people exposed to loud environments, such as the airport, construction sites, ambulances, and police vehicles, while allowing them to hear normal, natural sounds most of the time between loud noises. Since the devices can be made entirely of plastics without metals, they may be used inside magnetic resonance imaging (MRI) scanners, where the noise can be as high as 130 dB. With ergonomic designs, the technology has the potential to make ear protection more widely adopted in our daily lives, similar to how sunscreens protect our skin and photochromic sunglasses protect our eyes. Finally, besides their use as wearables, SHIELD filters can be incorporated in structures such as building walls for nonlinear blocking of noises.

### Fabrication of Devices

The nonlinear filters were fabricated at two different outer diameters: 9 mm for earplugs and 15 mm for earmuff and helmet prototypes. The effective area with open holes is 5.8 mm in diameter for the 9-mm filters and 10 mm for the 15-mm filters. The typical thickness of the filters is 2 mm. Grid pairs are fabricated using a 3D printer (model: Fomlabs Fom2). To achieve a precise gap size, grids are printed and then fine-polished to a typical thickness of 1 mm. Several pieces of thin spacers (such as pieces of aluminum foil) were glued on a flat grid, which determine the gap spacing between grids. Circular membranes are cut with a punch. After punching, the membrane is squeezed between two flat and smooth metallic plates to remove any surface curvatures, roughness, or wrinkles. The membrane is gently placed on a grid with spacers using a pair of tweezers, and the other grid is placed on top of the membrane to form the filter. It can be desirable to align the holes of the two grids. A small amount of low-viscosity superglue is applied to glue the two grids. To make earplugs, a cylindrical metallic frame was used with an inner diameter of 0.9 mm and a length of 1 cm. A 9-mm filter is inserted inside the tube. The filter is glued inside the frame closer to the distal end that goes inside in the ear to minimize the enclosed volume behind the filter and reduce sound distortion due to Helmholtz resonances. The earmuff and helmet prototypes use seven 15-mm filters in a honeycomb arrangement. The spacing between the filters and the ear is minimized and partially filled with soft foams to prevent acoustic resonances and minimize sound distortion.

### Acoustic Measurements

For characterizing the nonlinear transmission of devices, two microphones were used, one inside an isolating holder to measure the transmitted sound and the other to measure the input sound. Acoustic impulses are generated by popping balloons, with SPL peaks reaching up to 163 dB at 10 cm distance. Lower SPL peaks are achieved by adjusting the distance and air pressure of the balloons. The typical rise time of the impulses is around 200 μs. The data sampling rate is 44 kHz. Recordings of both microphones are compared to measure transmission. 5-10 impulses with identical SPL peaks are selected from the time domain data. Fast Fourier Transform (FFT) of the two microphones recordings is taken using the Audacity audio editor software. A time span of 9 seconds for each impulse is considered. The outcome is the spectral loss for that impulse. The spectral transmission loss is the average of the spectral losses for several impulses with the same SPL. The difference in spectral loss between impulses is small (<5 dB) for frequencies 0-4 kHz, but it increases with frequency. For 8 kHz, the difference is 5-10 dB. For low-SPL spectral analysis, a high sensitivity microphone was used to measure the transmitted sound through 14 the devices in real time. White noise with an intensity of 90 dB is generated by a cellphone speaker using noise sounds available in YouTube videos. After recording the transmitted sound, the device is removed, and the microphone records the reference signal in the absence of the device. Both recordings are then converted into spectral data using the FFT tool of the Audacity audio editor software. The spectral transmission loss of the device is obtained by subtracting the reference spectral data from the signal spectral data.

The present disclosure has described one or more preferred non-limiting examples, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the accompanying description or illustrated in the accompanying drawings. The disclosure is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular non-limiting examples or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or non-limiting examples. Further, references to particular rotational or other movements (e.g., counterclockwise rotation) is generally intended as a description only of movement relative a reference frame of a particular example of illustration.

In some non-limiting examples, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, non-limiting examples of the disclosure can be implemented as a set of instructions, tangibly embodied on a non-transitory computer-readable media, such that a processor device can implement the instructions based upon reading the instructions from the computer-readable media. Some non-limiting examples of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of the disclosure. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as non-limiting examples of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use non-limiting examples of the disclosure. Various modifications to the illustrated examples will be readily apparent to those skilled in the art, and the generic principles herein can be applied to other examples and applications without departing from the principles disclosed herein. Thus, non-limiting examples of the disclosure are not intended to be limited to non-limiting examples shown, but are to be accorded the widest scope consistent with the principles and features disclosed herein and the claims below. The accompanying detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of;” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g. “one or the other but not both”) when preceded by terms of exclusivity, such as “either,” “one of,” “only one of,” or “exactly one of.”

Also as used herein, unless otherwise specified or limited, the terms “about” and “approximately,” as used herein with respect to a reference value, refer to variations from the reference value of ±15% or less (e.g., ±10%, ±5%, etc.), inclusive of the endpoints of the range. Similarly, the term “substantially equal” (and the like) as used herein with respect to a reference value refers to variations from the reference value of less than ±30% (e.g., ±20%, ±10%, ±5%) inclusive. Where specified, “substantially” can indicate in particular a variation in one numerical direction relative to a reference value. For example, “substantially less” than a reference value (and the like) indicates a value that is reduced from the reference value by 30% or more, and “substantially more” than a reference value (and the like) indicates a value that is increased from the reference value by 30% or more.

Various features and advantages of the disclosure are set forth in the following claims.

1. An acoustic filter comprising:
a first substrate including a first plurality of holes directed therethrough;
a second substrate including a second plurality of holes directed therethrough;
a chamber defined between the first substrate and the second substrate;
a membrane positioned within the chamber, the membrane having a dimension other than the thickness of the membrane that is less than a corresponding dimension of the chamber;
wherein the acoustic filter is configured to attenuate a first acoustic wave that passes through the acoustic filter, the first acoustic wave having a first amplitude above an amplitude threshold; and
wherein the acoustic filter is configured to passthrough a second acoustic wave without substantially attenuating the second acoustic wave, the second acoustic wave having a second amplitude below the amplitude threshold.

2. The acoustic filter of claim 1, wherein a gap is between a peripheral end of the membrane and the chamber.

3. The acoustic filter of claim 2, wherein the gap is between the peripheral end of the membrane and the top of the chamber; or
wherein the gap is between the peripheral end of the membrane and the bottom of the chamber.

4. The acoustic filter of claim 1, wherein the dimension of the membrane other than the thickness of the membrane is a length, a width, or a diameter.

5. The acoustic filter of claim 1, wherein the membrane has a length that is less than a length of the chamber, such that a first longitudinal end of the membrane does not contact the first substrate or the second substrate.

6. The acoustic filter of claim 5, wherein a second longitudinal end of the membrane opposite the first longitudinal end of the membrane does not contact the first substrate or the second substrate.

7. The acoustic filter of claim 1, wherein the length of the membrane is smaller than a length of the first substrate; and
wherein the length of the membrane is smaller than a length of the second substrate.

8. An acoustic filter comprising:
a first substrate including a first plurality of holes directed therethrough;
a second substrate including a second plurality of holes directed therethrough;
a chamber defined between the first substrate and the second substrate;
a membrane positioned within chamber, wherein the membrane is unconstrained or is not taut between opposing ends of the membrane prior to an acoustic wave being applied to the acoustic filter;
wherein the acoustic filter is configured to attenuate a first acoustic wave that passes through the acoustic filter, the first acoustic wave having a first amplitude above an amplitude threshold; and
wherein the acoustic filter is configured to passthrough a second acoustic wave without substantially attenuating the second acoustic wave, the second acoustic wave having a second amplitude below the amplitude threshold.

9. The acoustic filter of claim 8, wherein a gap is between a top of the membrane and a top of the chamber; or
wherein a gap is between a bottom of the membrane and the bottom of the chamber.

10. The acoustic filter of claim 8, wherein a first end of the membrane is not coupled to the first substrate or the second substrate;
wherein a second end of the membrane opposite the first end is not coupled to the first substrate or the second substrate; and
wherein a center of the membrane between the first end and the second end is not coupled to the first substrate or the second substrate.

11. The acoustic filter of claim 8, wherein the membrane is free to translate along a longitudinal axis of the acoustic filter that is substantially parallel to a longitudinal dimension of the membrane.

12. The acoustic filter of claim 8, wherein the membrane being unconstrainted or not taut avoids substantially adding an acoustic impedance to the acoustic filter that results in acoustic losses for sound transmitted therethrough.

13. The acoustic filter of claim 8, wherein the acoustic filter is configured to passively attenuate the first acoustic wave, such that no electrical power source is needed.

14. The acoustic filter of claim 8, wherein each hole of the first plurality of holes is aligned with a respective hole of the second plurality of holes.

15. The acoustic filter of claim 8, wherein the chamber includes a first width at a first end of the chamber, a second width at a second end of the chamber, and a third width at a center of the chamber; and
wherein the first width, the second width, and the third width are substantially the same.

16. The acoustic filter of claim 8, wherein a cross-section of the chamber is rectangular or square.

17. The acoustic filter of claim 8, wherein the width of the chamber is substantially uniform along the entire length of the chamber.

18. The acoustic filter of claim 8, wherein the first amplitude of the first acoustic wave is greater than or equal to 70 dB; and
wherein the second amplitude of the second acoustic wave is less than 70 dB.

19. The acoustic filter of claim 18, wherein the first amplitude of the first acoustic wave is greater than or equal to 120 dB.

20. The acoustic filter of claim 19, wherein the first amplitude of the first acoustic wave is greater than or equal to 150 dB; or
wherein the first amplitude of the first acoustic wave is greater than or equal to 170 dB.

21. The acoustic filter of claim 8, wherein a width of the chamber is less than 3 millimeters;
wherein the width of the chamber is less than 2 millimeters;
wherein the width of the chamber is less than 1 millimeter; or
wherein the width of the chamber is less than 0.6 millimeters.

22. The acoustic filter of claim 8, wherein each hole of the first plurality of holes has a size that is less than or equal to 7.5 millimeters or 0.5 millimeters; and
wherein each hole of the second plurality of holes has a size that is less than or equal to 7.5 millimeters or 500 millimeters.

23. The acoustic filter of claim 8, wherein the first substrate is rigid or semi-rigid; and
wherein the second substrate is rigid or semi-rigid.

24. The acoustic filter of claim 8, wherein the first amplitude of the first acoustic wave is attenuated by at least 30 dB.

25. The acoustic filter of claim 8, wherein the first substrate includes a first recess fluidly coupled to the first plurality of holes;
wherein the second substrate includes a second recess fluidly coupled to the second plurality of holes; and
wherein the first recess and the second recess define the chamber.

26. The acoustic filter of claim 8, wherein the membrane does not include a hole directed therethrough;
wherein the membrane has a planar surface, such that the membrane is substantially flat; and
wherein the membrane has a surface that is substantially smooth.

27. An acoustic filter comprising:
a first substrate including a first plurality of holes directed therethrough;
a second substrate including a second plurality of holes directed therethrough;
a chamber defined between the first substrate and the second substrate, the chamber having a first width at a top of the chamber and a second width at a center of the chamber, the first width and the second width being substantially the same;
a membrane positioned within the chamber;
wherein the acoustic filter is configured to attenuate a first acoustic wave that passes through the acoustic filter, the first acoustic wave having a first amplitude above an amplitude threshold; and
wherein the acoustic filter is configured to passthrough a second acoustic wave without substantially attenuating the second acoustic wave, the second acoustic wave having a second amplitude below the threshold.

28. The acoustic filter of claim 27, wherein each hole of the first plurality of holes and the second plurality of holes has a size that is less than 0.5 millimeters.

29. The acoustic filter of claim 23, wherein the first width of the chamber is less than or equal to 300 μm; or
wherein the second width of the chamber is less than or equal to 300 μm.

30. (canceled)

31. A sensor assembly comprising:
an acoustic transducer that is configured to convert acoustic waves into electrical signals;
an acoustic filter fluidly coupled to the acoustic transducer, wherein the acoustic filter is positioned in front of the acoustic transducer, such that an acoustic wave propagates first through the acoustic filter and then to the acoustic transducer; and
wherein the acoustic filter includes:
a first substrate including a first plurality of holes directed therethrough;
a second substrate including a second plurality of holes directed therethrough;
a chamber defined between the first substrate and the second substrate;
a membrane positioned within chamber;
wherein the acoustic filter is configured to attenuate a first acoustic wave that passes through the acoustic filter, the first acoustic wave having a first amplitude above an amplitude threshold; and
wherein the acoustic filter is configured to passthrough a second acoustic wave without substantially attenuating the second acoustic wave, the second acoustic wave having a second amplitude below the amplitude threshold.

32. The sensor assembly of claim 31, wherein the acoustic transducer is a microphone sensor; and
wherein the sensor assembly is a microphone.

33. A hearing aid comprising:
the sensor assembly of claim 31.

34. A hearing device comprising:
an acoustic transducer that is configured to convert electrical signals into acoustic waves;
an acoustic filter configured to be fluidly coupled to an ear canal, the acoustic filter including:
a first substrate including a first plurality of holes directed therethrough;
a second substrate including a second plurality of holes directed therethrough;
a chamber defined between the first substrate and the second substrate;
a membrane positioned within chamber;
wherein the acoustic filter is configured to attenuate a first acoustic wave that passes through the acoustic filter, the first acoustic wave having a first amplitude above an amplitude threshold; and
wherein the acoustic filter is configured to passthrough a second acoustic wave without substantially attenuating the second acoustic wave, the second acoustic wave having a second amplitude below the threshold.

35. The hearing device of claim 34, wherein the acoustic filter is positioned in front of the acoustic transducer, such that an acoustic wave generated from the acoustic transducer propagates through the acoustic filter and then to into the ear canal; or
wherein the acoustic filter is positioned behind the acoustic transducer, such that an acoustic wave generated from the acoustic transducer avoids passing through the acoustic filter before entering the ear canal.

36. The hearing device of claim 34, wherein the hearing device is a hearing aid.

37. An acoustic filter comprising:
a first substrate including a first plurality of holes directed therethrough;
a second substrate including a second plurality of holes directed therethrough;
a membrane positioned between the first substrate and the second substrate, the membrane having a dimension other than the thickness of the membrane that is less than a corresponding dimension of the first substrate or the second substrate; and
wherein the acoustic filter is configured to attenuate a first acoustic wave that passes through the acoustic filter, the first acoustic wave having a first amplitude above an amplitude threshold.

38. The acoustic filter of claim 37, wherein the acoustic filter is configured to passthrough a second acoustic wave without substantially attenuating the second acoustic wave, the second acoustic wave having a second amplitude below the amplitude threshold.

39. The acoustic filter of claim 37, wherein the amplitude threshold is less than or equal to 115 dB.
